(19) United States
(12) Patent Application Publication
(10) Pub. No.: US 2006/0125475 A1
(43) Pub. Date: Jun. 15, 2006

(54) Radio frequency impedance mapping

(76) Inventors: Daniel K. Sodickson, Newton, MA (US); Aaron K. Grant, Allston, MA (US)

Correspondence Address: WOLF GREENFIELD & SACKS, PC;FEDERAL RESERVE PLAZA, 600 ATLANTIC AVENUE, BOSTON, MA, 02210-2206, US

(21) Appl. No.: 10/527,592

(22) PCT Filed: Sep. 17, 2003
(86) PCT No.: PCT/US03/29533

§ 371 Date: Jan. 23, 2006

Related U.S. Application Data
(60) Provisional application No. 60/411,450, filed on Sep. 17, 2002.

(51) Int. Cl. G01V 3/00; A61B 5/05; H01F 5/00

(52) U.S. Cl. 324/300; 324/307; 600/416; 600/425; 335/299

(57) ABSTRACT

Methods and apparatus for determining dielectric properties such as conductivity, permittivity and/or permeability of a body are provided. An array of resonant coils (950) capable of generating electromagnetic radiation are provided proximate a body (950) to imaged. Properties of the array of coils are influenced by the loading effect of the body. More particularly, one or more resonant properties of the coils are perturbed, the perturbation of which may be measured to obtain an indication of the conductivity, permittivity and permeability of the loading body.

### CROSS-REFERENCE TO RELATED APPLICATION

This application claims the benefit under 35 U.S.C. §119(e) of U.S. Provisional Application 60/411,450, filed Sep. 17, 2002, entitled “RADIO FREQUENCY IMPEDANCE MAPPING FOR MEDICAL IMAGING,” by Sodickson et. al, which is incorporated herein in its entirety.

### FIELD OF THE INVENTION

The present invention relates generally to non-invasive imaging applications, for example, MRI. More particularly, the present invention relates to imaging techniques employing radio frequency (RF) coils to measure properties of a body being imaged.

### BACKGROUND OF THE INVENTION

A variety of imaging techniques have been employed to generate images of the internal characteristics of an object in diverse applications ranging from medical imaging to detection of prohibited materials in baggage at security checkpoints. For example, magnetic resonance imaging (MRI), X-ray computed tomography (CT), ultrasound, etc., have been widely utilized in medical applications to image the internal structures of an object, such as a patient. Generally, an imaging modality takes advantage of technology that facilitates discriminating portions of an object based on one or more properties or characteristics of the object that can be measured.

For example, X-ray CT includes measuring the attenuation of electromagnetic radiation as it passes through an object. The resulting images carry information about the X-ray absorption characteristics of material within the object, which is related to the atomic number of the material. MRI measures various magnetic properties such as relaxation times associated with various spin characteristics (e.g., realignment with an axis of precession) of material in a magnetic field. Ultrasound measures a material's capacity to reflect sound waves.

While these techniques have been successful in discriminating between different materials (e.g., tissue, bone, etc.) to form images based on respective properties that can be isolated and measured, there may be subject matter of interest that cannot be readily distinguished when characterized according to the particular properties exploited by a respective conventional imaging modality. For example, a tumor may have substantially the same X-ray absorption characteristics as the surrounding tissue, rendering the tumor effectively “invisible” to X-ray CT.

Further, drawbacks of conventional imaging modalities such as X-ray CT and MRI include various practical limitations on the equipment. MRI and X-ray CT devices are generally immobile and relatively expensive, often requiring dedicated facilities for operation. The general bulk of such systems may prohibit the technology from being transported to a body, preventing these modalities from being employed in the field or in emergency situations when a body (e.g., a patient) cannot be transported to the facility.

Other imaging techniques such as electrical impedance tomography (EIT) have illustrated that dielectric properties (e.g., conductivity, permittivity, etc) of a body may be a viable discriminating property to obtain images of internal structures of a body. In EIT, electrical currents are provided to the body through a set of electrodes applied to the surface of the body, for example, the skin of a patient. Changes in electric potential at each electrode are measured to determine dielectric properties of the body. However, EIT requires that electrical currents be applied directly to the body and that these currents be able to pass throughout the entire volume being imaged. Accordingly, some regions of a body may be difficult to image. For example, in human bodies, high resistance regions such as the skull may prevent brain images from being obtained. In addition, measurement uncertainties arise due to the impedance at the interface between the electrodes and the surface of the body being imaged.

In magnetic induction tomography (MIT), a pair of solenoid coils or gradiometer coils are positioned near an object to be imaged. The solenoid coils may operate as excitation coils and/or sensing coils. An excitation coil generates an oscillating magnetic field which is, in turn, detected by the sensing coil via magnetic inductance properties. The presence of a dielectric body between an excitation coil and a sensing coil perturbs the magnetic field sensed by the sensing coil. Magnetic field perturbation results in a change in the mutual inductance between excitation and sensing coils (often measured as a phase shift in the magnetic field caused by eddy currents induced in the dielectric object). Each excitation/sensing coil pair may provide a ‘projection’ of magnetic field perturbation. These projections may then be employed in various tomography techniques such as back-projection to reconstruct an image of the dielectric properties of the object.

However, MIT considers predominantly the properties of mutual inductance, for example, changes caused by eddy currents resulting from current induced in the dielectric object. MIT utilizes solenoid coils or loop gradiometer pairs responsive predominantly to inductive coupling information. In general, loading effects on the inductive coupling between solenoid or gradiometer pairs are quite small and relatively difficult to measure. For example, a very precise measurement (on the order of a part in 1,000,000 or better) may be required to detect changes in inductive coupling resulting from the presence of a dielectric body. In addition, MIT relies on back-projection techniques which have been shown to yield quantitatively inaccurate results.

Despite the shortcomings of imaging modalities such as EIT and MIT, the dielectric properties of a body remain a viable characteristic for discriminating between regions of the body. For example, tumors generally have elevated values of both conductivity and permittivity relative to the surrounding tissue. Hepatomas in rats, cancerous tissues in the breast, lung, colon, kidney and liver have been shown to have heightened dielectric properties relative to the tissue from which the cancer was derived. In addition, cerebral edema, spreading depression, myocardial ischemia and other pathologies may also exhibit dielectric contrast to the surrounding tissues and may benefit from imaging modalities capable of detecting this dielectric contrast (e.g., contrast in a material's conductivity and/or permittivity characteristics).

As discussed above, MRI detects spin characteristics of target material to be imaged. MRI includes aligning the spin of nuclei of material being imaged in a generally homogeneous magnetic field and perturbing the magnetic field with periodic radio frequency (RF) pulses in order to measure the nuclear magnetic resonance (NMR) phenomenon of the material being imaged. To invoke the NMR phenomenon, one or more resonant coils are provided that generate the RF pulses at a resonant frequency that matches a Larmor frequency (i.e., the rate at which a nucleus precesses about an axis) of certain tissue in order to excite the nuclei such that they precess about an axis in the direction of the applied RF pulse. When the RF pulse subsides, the nuclei realign with the magnetic field, releasing energy that can be measured.

However, when a resonant coil is placed in proximity of a load, for example, a patient or other object to be imaged, various properties of the resonant coil may be affected. In MRI, this loading effect tends to negatively impact the operation of the device by altering the resonant frequency of the coil and causing other generally undesirable changes in the in coil properties. This loading effect depends in part on the dielectric properties of the load. Changes in resonant frequency of the coil may reduce the device's ability to excite the nuclei of the material being imaged (e.g., by creating a mismatch between the coil's resonant frequency and the Larmor frequency of the target material) and negatively impact the quality of the resulting images. The effects of coil loading complicate MRI to the extent that resonant coils are often tuned or adjusted to compensate for the generally undesirable loading effect caused by the body being imaged.

### SUMMARY OF THE INVENTION

One embodiment according to the present invention includes a method of determining dielectric properties of a body positioned proximate an array of coils having one or more resonant properties, the method comprising acts of detecting a change in at least one resonant property of at least one of the coils in the array, determining at least one electromagnetic property of at least one region of the body from the change in the at least one resonant property.

Another embodiment according to the present invention includes a method of determining dielectric properties of a body, the method comprising acts of positioning the body proximate a plurality of coils, measuring at least one property of at least one of the plurality of coils, and determining at least one electromagnetic property of at least one region of the body from the at least one property based on at least two of a resistive coupling, a capacitive coupling, and an inductive coupling between at least two of the plurality of coils.

Another embodiment according to the present invention includes an apparatus for determining dielectric properties of a body. The apparatus comprises a plurality of coils having one or more resonant properties, a first component coupled to the plurality of coils and adapted to provide at least one measurement of the plurality of coils indicative of a change in at least one resonant property of at least one of the plurality of coils, and a second component coupled to the first component to receive the at least one measurement, the second component adapted to determine at least one electromagnetic property of at least one region of the body based on the change in the at least one resonant property.

Another embodiment according to the present invention include an apparatus for determining dielectric properties of a body. The apparatus comprises a plurality of coils, a first component coupled to the plurality of coils, the first component adapted to provide at least one measurement of at least one property of the plurality of coils, and a second component coupled to the first component to receive the at least one measurement, the second component adapted to determine at least one electromagnetic property of at least one region of the body from the at least one measurement based on at least two of a resistive coupling, a capacitive coupling, and an inductive coupling between two or more of the plurality of coils.

Another embodiment according to the present invention includes a computer readable medium encoded with instructions capable of being executed on at least one processor, the instructions, when executed by the at least one processor, performing acts comprising defining an electromagnetic model of the coil array, receiving an input including a measured impedance matrix of the coil array, logically partitioning a volume associated with the model of the coil array and the body into a plurality of regions, assigning trial values respectively to each of the plurality of regions, the trial values including at least one of conductivity, permittivity and permeability, generating a trial impedance matrix from the assigned trial values according to the electromagnetic model of the coil array, and reducing a distance between the trial impedance matrix and the measured impedance matrix.

### BRIEF DESCRIPTION OF THE DRAWINGS

FIG. 1 illustrates a loading effect by mutual inductance between a conducting loop and a dielectric loop;

FIG. 2 illustrates one embodiment according to the present invention of a resonant coil suitable for arranging in an array to determine dielectric properties of a body to be imaged;

FIG. 3 illustrates one embodiment according to the present invention of a method for determining dielectric properties of a body by measuring the loading effect of the body on a coil array;

FIGS. 4A and FIGS. 4B illustrate how spatial information on the dielectric distribution of a volume may be obtained using couplings of magnetic flux between coils in an array;

FIG. 5 illustrates one embodiment according to the present invention of a method for determining dielectric properties of a body being imaged by measuring an impedance matrix of a coil array;

FIG. 6 illustrates a coil array and a body that may be employed in connection with the method illustrated in FIG. 5;

FIG. 7 illustrates one embodiment according to the present invention for calibrating a coil array;

FIG. 8 illustrates one embodiment according to the present invention of a coil array and a schematic of a model of the array;

FIG. 9 illustrates one embodiment according to the present invention of an image acquisition system adapted to obtain one or more RFIM images;

FIGS. 10A, 10B and 10C illustrate several embodiments of matching circuitry that may be used to measure properties of a coil array according to various aspects of the present invention;

FIG. 11 illustrates one embodiment according to the present invention of a method of acquiring one or more RFIM images;

FIGS. 12A and FIGS. 12B illustrate one embodiment according to the present invention of an arrangement of a coil array; and

FIG. 13 illustrates another embodiment according to the present invention of an arrangement of a coil array.

### DETAILED DESCRIPTION

While loading effects may cause generally undesirable results in conventional imaging modalities such as MRI, Applicant has recognized that the sensitivity of resonant coils to loading effects may be exploited to effectively measure dielectric properties of a body proximate to the coil. In particular, Applicant has identified and appreciated that a change in resonant properties of one or more resonant coils due to loading may provide information about the distribution of dielectric characteristics of the loading body.

The term “resonant property” refers to a characteristic, trait or feature indicative of the resonance of a coil including, but not limited to, resonant frequency, phase, decay characteristics, lineshape, etc. The term “loading effect” refers generally to any change in the electrical or magnetic property of an resonant coil resulting from the presence of a body. For example, the loading effect may include, but is not limited to, changes in the resonant properties of the coil, electromagnetic fields generated by the coil, coil impedance, etc.

In one embodiment according to the present invention, changes in resonant properties of one or more resonant coils may be measured to determine the dielectric properties of a body. For example, impedance characteristics of one or more coils resulting from shifts in the resonant frequency of the coils may be measured to determine the distribution of dielectric characteristics of a loading body. Dielectric properties or characteristics refer generally to electrical conductivity, electrical permittivity and/or magnetic permeability (also referred to simply as conductivity, permittivity and permeability, respectively). The term “body” will be used herein interchangeably with the term “object” to refer generally to any mass capable of modifying one or more properties of a resonant coil, for example, by acting as a load.

A resonant coil typically resonates at frequencies within the radio frequency (RF) spectrum. The RF spectrum is typically considered to span frequencies from approximately 3 kHz to 100 GHz. However, a resonant coil may be made to resonate outside this range. A resonant coil may be characterized by an inductive-capacitive-resistive (LCR) circuit. For example, a resonant coil may be comprised of one or more series capacitors connected by conducting material to form a loop having an inductance L, a resistance R, and a capacitance C. Accordingly, the impedance of such a coil at a frequency ω may be expressed as,
Z₀(ω)=iωL−i/ωC+R (1)

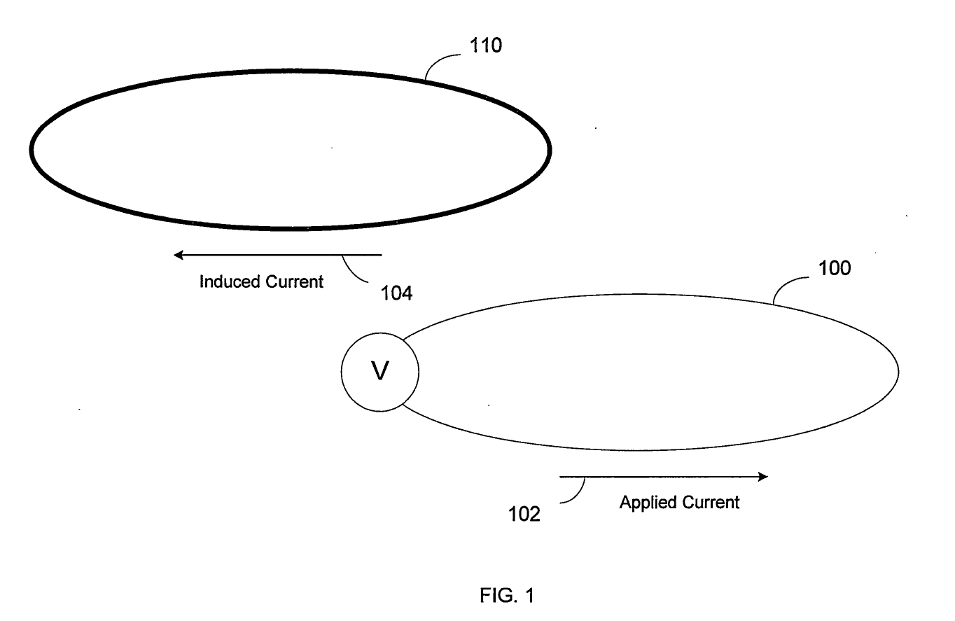

When such a coil is placed near a loading body, the impedance characteristics of the coil may be affected. Changes in impedance characteristics may be measured to obtain information about the dielectric make-up of the body loading the coil. For example, a body acting as a load on the coil may be characterized as one or more dielectric loops as illustrated in FIG. 1. Dielectric body 110 may also be considered as an LCR circuit having an inductance l, capacitance c and resistance r. The values of l, c and r may depend on the dielectric properties of the body such as conductivity and permittivity.

When dielectric body 110 is placed proximate to a resonant coil 100 and the coil is operated, the resonant electromagnetic (EM) field generated by the coil 100 induces a current in dielectric loop 110, which in turn effects the properties of the resonant coil. For example, when a voltage V is provided to coil 100 such that an applied current 102 is generated in coil 100, dielectric body 110 experiences an induced current 104. Accordingly, the resonant coil and dielectric loop inductively couple, that is, they exhibit a mutual inductance. In the presence of a dielectric body, the impedance of the RF coil at frequency ω may be expressed as,
Z(ω)=Z₀(ω)+ω²M²/(iωl−i/ωc+r) (2)

That is, the impedance of the coil is affected by the presence of the load and depends in part on the dielectric properties of the loading body. Equation 2 provides one description of the impedance of a resonant coil in terms of the properties of a dielectric body.

Applicant has identified and developed methods of determining dielectric properties of a body from the impedance characteristics of one or more resonant coils operated in the presence of the body. The term “operate” with respect to a coil, refers generally to directly generating a current (e.g., from a power source) in the conductive loop of the coil. For example, a voltage or current source may be provided at an input to operate the coil. The term “radio frequency impedance mapping” (RFIM) will be used herein to describe generally any of various methods of mapping impedance characteristics of one or more resonant coils to dielectric properties of a body coupled to the one or more resonant coils.

When a resonant property of a coil changes, for example, due to the presence of a loading body, the impedance characteristics of the coil experience a corresponding change. In some exemplary coils, a resistance may change from 0.5 Ohms on resonance when the coils are not loaded to 3 Ohms or more when loaded. In addition, eddy currents and displacement currents may modify the inductance and/or capacitance of such coils by an amount sufficient to shift a resonant frequency of a respective coil by one percent or more.

Accordingly, changes in resonant properties of an array of coils due to the presence of a loading body may be determined by measuring the impedance characteristics of the array to obtain information about the dielectric distribution of the body. When a coil is operated at its resonant frequency, an impedance of the coil is quite small and loading effects may be quite large by comparison. As a result, changes in resonant properties may facilitate measurement precision that is far less demanding than other techniques, for example, MIT.

The term “impedance characteristics” refers generally to measurements from which impedance may be derived. For example, impedance characteristics may be direct impedance measurements, resistance values, or may be measurements of scattering parameters (S-parameters) which can be converted to and from impedance values. The term “array” refers generally to a plurality of coils arranged in a generally known relationship to one another.

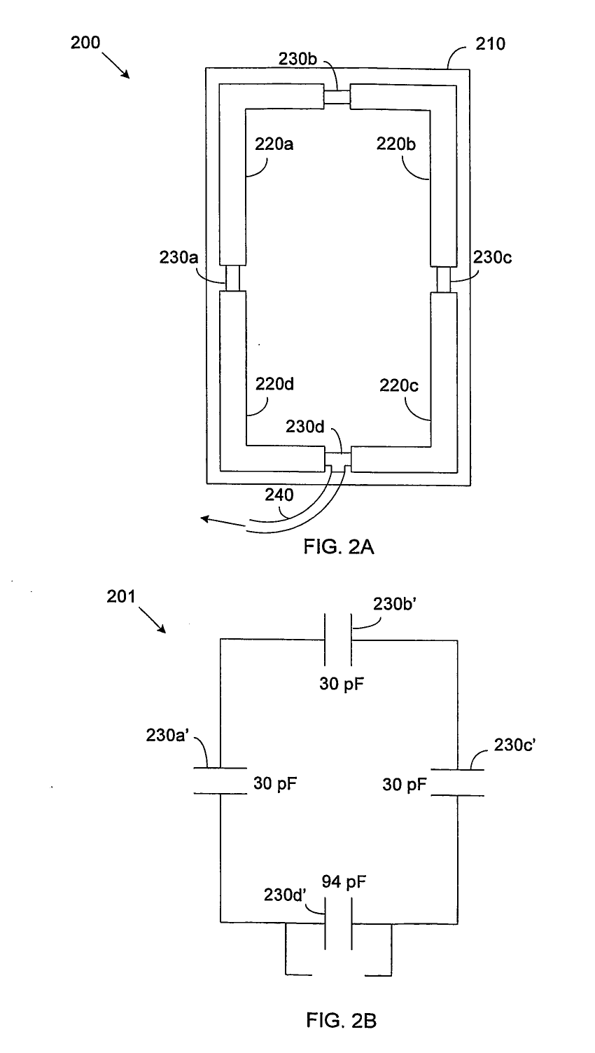

It should be appreciated from the foregoing that determining dielectric properties of a body may include measuring one or more impedance characteristics of a coil array. FIG. 2A illustrates one embodiment according to the present invention of a resonant coil that may be arranged in an array and employed to obtain dielectric properties of a loading body. Coil 200 may be a substantially planar coil capable of radiating electromagnetic energy. Coil 200 may include a generally non-conductive substrate 210 on which to mount a conducting loop. The conducting loop may comprise a plurality of conductive strips 220a-220d coupled together by capacitors 230a-230d. The conductive strips and the capacitors together form a resonant LCR circuit. The conductive strips may be formed of, for example, copper material or any other conductive metal or substance.

Coil 200 may be constructed, for example, from a printed circuit board (PCB) board having a layer of copper over a substrate of a plastic material or other insulative material. The copper may then be etched to form the conductive loop. Coil 200 may also be constructed by applying copper strips to an adhesive substrate, or by any other method suitable for providing a conductive loop. A coil constructed of multiple turns or layers of conductor may be used. Capacitors 230a-230d may be any of a variety or combination of lumped elements or, in some embodiments, may be suitable distributed elements. Numerous combinations and variations in implementing a conductive loop will occur to those skilled in the art. The variety of methods of implementing a resonant circuit is considered to be within the scope of the invention.

One of the capacitors (e.g., capacitor 230d) may be a matching capacitor. The matching capacitor may be used to match the input impedance of the coil to an RF power source. Matching capacitor 230d may be placed between leads of a connector adapted to couple to a power source capable of generating a current in the conductive loop of the coil. For example, coil 200 may be connected to a voltage source capable of providing a broadband electric field pulse via a Bayonet Neill Concelman (BNC) cable or other suitable coaxial cable 240. Matching capacitor 230d may be chosen so that the coil has an input impedance that matches the impedance of the cable (e.g., a 50 Ohm or 75 Ohm cable). It may be desirable to match the input impedance of the coil at the nominal resonant frequency of the coil. It may also be desirable to match the input impedance of the coil at its resonant frequency when it is loaded by a body having similar characteristics to the body to be imaged.

The term “nominal” in connection with a resonant frequency refers to the frequency at which the coil was adapted to resonate. In particular, the nominal frequency refers to the resonant frequency of an unloaded coil. As such, a change in resonant frequency refers to the change from the nominal value of the resonant frequency of the coil. Likewise, a shift in resonant frequency refers to a change from a nominal resonant frequency that may occur, for example, when a resonant coil is loaded.

The nominal resonant frequency of coil 200 depends in part on the arrangement and values of capacitors 230a-230d. FIG. 2B illustrates a circuit schematic of one embodiment of a resonant coil according to the present invention. Circuit 201 may schematically represent a coil similar to coil 200 having capacitance values as indicated. Specifically, capacitors 230a′-230c′ may be 30 pF capacitors, and the matching capacitor 230d′ may be a 94 pF capacitor. A coil arranged substantially as shown in FIG. 2B may have a resonant frequency of approximately 110 MHz. It should be appreciated that coils configured to have different nominal resonant frequencies may have differently valued capacitances. Similarly, coils having a nominal resonant frequency of 110 MHz may be designed using components different from the exemplary coil described above.

Various properties of the loading effect may be exploited using resonant coils that resonate at any number of frequencies. At higher resonant frequencies, for example, at frequencies approaching 1 GHz, the loading effect is relatively strong. However, the electromagnetic fields generated at such high frequencies may have difficulty penetrating material comprising the body to be imaged. For example, when the body to be imaged is a human patient, high frequency EM fields may have difficulty penetrating tissue due in part to the so-called “skin depth” effect.

At relatively low frequencies, for example, frequencies well below 100 MHz, the contrast between dielectric properties of a body may be enhanced. However, at relatively low frequencies the loading effect tends to becomes less significant and more difficult to measure. However, any resonant frequency may be used and may be chosen in consideration of the properties of the body being imaged and the characteristics of a given coil or coil array.

In one embodiment, coil capacitors (e.g., capacitors 230a-230d) may be replaced with varactors having adjustable capacitance. Accordingly, a coil may be provided having a variable resonant frequency. Such a coil array may be adjusted to resonate at one or more different frequencies in consideration of the type of body being imaged, the arrangement of coils, or other considerations of the imaging environment.

It should be appreciated that FIGS. 2A and 2B illustrate only one of many varieties of resonant coils that may be suitable for practicing various aspects of the present invention. Other coils may be suitable, including, but not limited to, birdcage coils, surface coils, volume coils, transmission line array coils, various other resonant coils, etc. A resonant coil may comprise any number and arrangement of reactive components (e.g., capacitors and inductors) by which resonant electromagnetic fields may be generated.

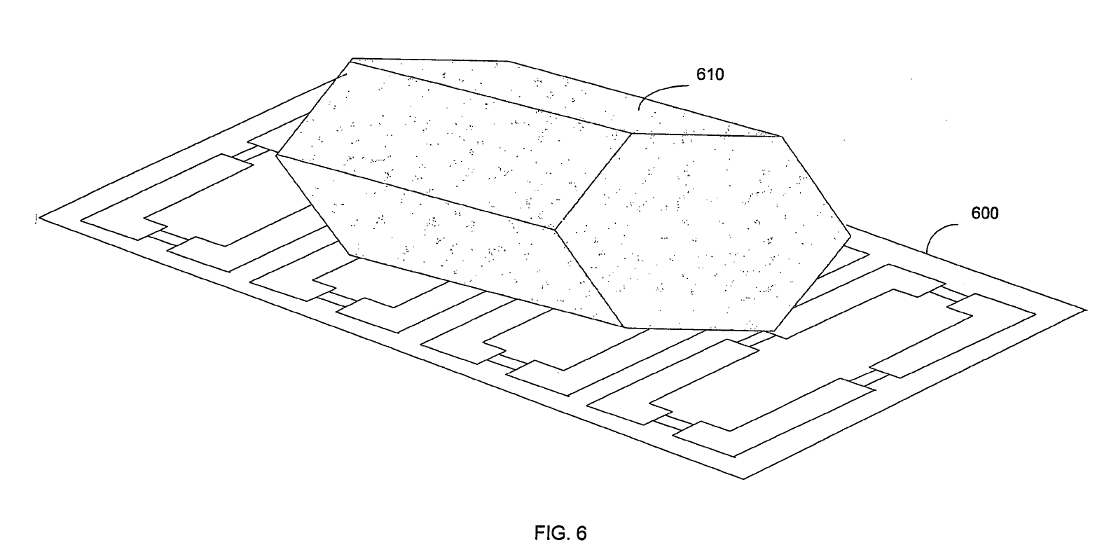

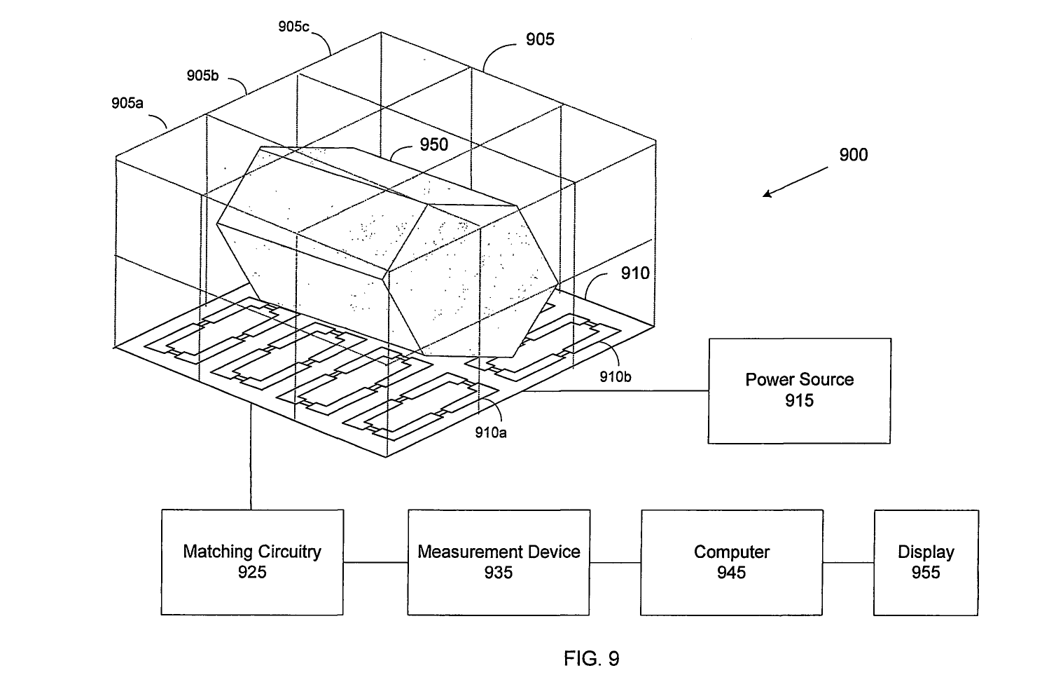

For simplicity and clarity, various aspects of the present invention will be illustrated in connection with the generally planar coil illustrated in FIG. 2. For example, FIGS. 6 and 9 illustrate coil arrays comprising a plurality of coils schematically depicted as the general conductive loop connected by lumped capacitors. However, this depiction is meant to indicate the presence of a resonant coil and not the specific type or arrangement (e.g., planar, birdcage, etc.). As such, any type of resonant coil could be used. The invention is not limited to the coils specifically illustrated herein and contemplates use with any of the coils mentioned above or any other coils capable of generating resonant EM fields.

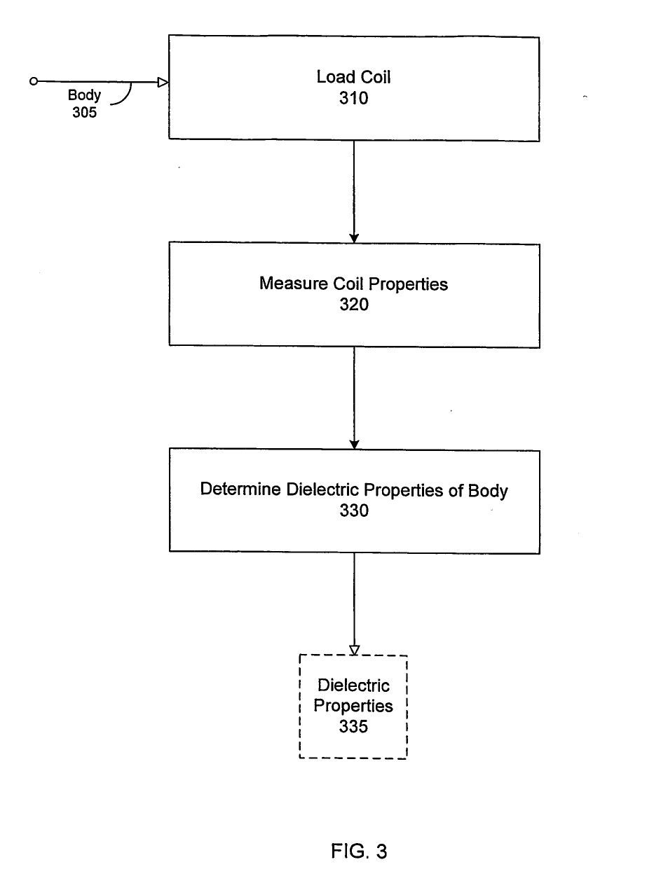

FIG. 3 illustrates one embodiment according to the present invention of a method for determining dielectric properties of a body acting as a load on an array of RF coils. The coil array may comprise a plurality of coils each having a resonant frequency. That is, an unloaded coil may be configured to resonate at a generally known nominal frequency. Each coil in the array may be designed to have a same or different nominal resonant frequency.

In step 310, a body (step 305) to be imaged may be positioned proximate to the plurality of coils in the array. In particular, the body is placed in a spatial relationship with the array such that the body acts as a load, causing some measurable loading effect on the array. The body is typically positioned in a generally known relationship with the array. The body may comprise one or more regions with unknown dielectric properties. For example, the body may be a patient having regions of homogeneous and inhomogeneous regions of conductivity and/or permittivity, or the body may be baggage such as airline luggage to be analyzed for items having certain conductivity and/or permittivity characteristics.

In step 320, a change in one or more resonant properties the coil array may be detected. For example, one or more measurements of properties of the coil array may be obtained. As described above, the loading effect resulting from placing the load proximate to the one or more coils will affect various properties of the coils. For example, various impedance characteristics of the coil array may be measured by operating one or more of the coils and measuring the impedance characteristics of one or more other coils in the array.

In step 330, the change in the one or more resonant properties of at least one of the coils may be used to determine dielectric properties of the body. For example, one or more properties measured from the coil array may be employed to determine the dielectric properties of the body. Measured impedance characteristics may indicate properties of the load, for example, as expressed in the equation 1. Impedance characteristics of a loaded coil array may encode information about resonant frequency shifts, and resistive, capacitive, and inductive coupling between coils in the array that can be used to determine the dielectric distribution of the load.

It may be desirable to form one or more images of the dielectric properties obtained from a body. An image typically represents intensity as a function of space. The term “intensity” refers generally to a magnitude, degree and/or value at some location in the image, which in turn corresponds to a respective region of space.

For example, in an X-ray image, intensity generally represents the absorption characteristics (e.g., density) of scanned material at a particular location in space. An image (e.g., an RFIM image) obtained via methods according to the present invention may have intensity values representing conductivity, permittivity, permeability and/or other electromagnetic characteristics (e.g., electric and/or magnetic field magnitude, direction, phase, or some combination of the above) as a function of space or may have an intensity representing some combination of one or more of the above. In a two dimensional image, each intensity value may correspond to an area in space and is referred to as a pixel. In a three dimensional image, each intensity value may correspond to a volume in space and is referred to as a voxel.

Accordingly, an RFIM image may involve determining spatial information about the dielectric properties of a body, that is, a dielectric distribution of the body may be obtained from impedance measurements of the one or more resonant coils. In one embodiment according to the present invention, an array of resonant coils is employed to encode spatial information about the dielectric properties of a body.

In another embodiment, a single coil may be used to determine dielectric properties of the body. The single coil may be translated, rotated or otherwise varied in location with respect to the body being imaged. One or more properties of the single resonant coil may be measured at each of the locations. Accordingly, each placement of the coil will provide information about the overlap of the coil's electromagnetic field with the dielectric variation inside the body to be imaged as described in further detail below.

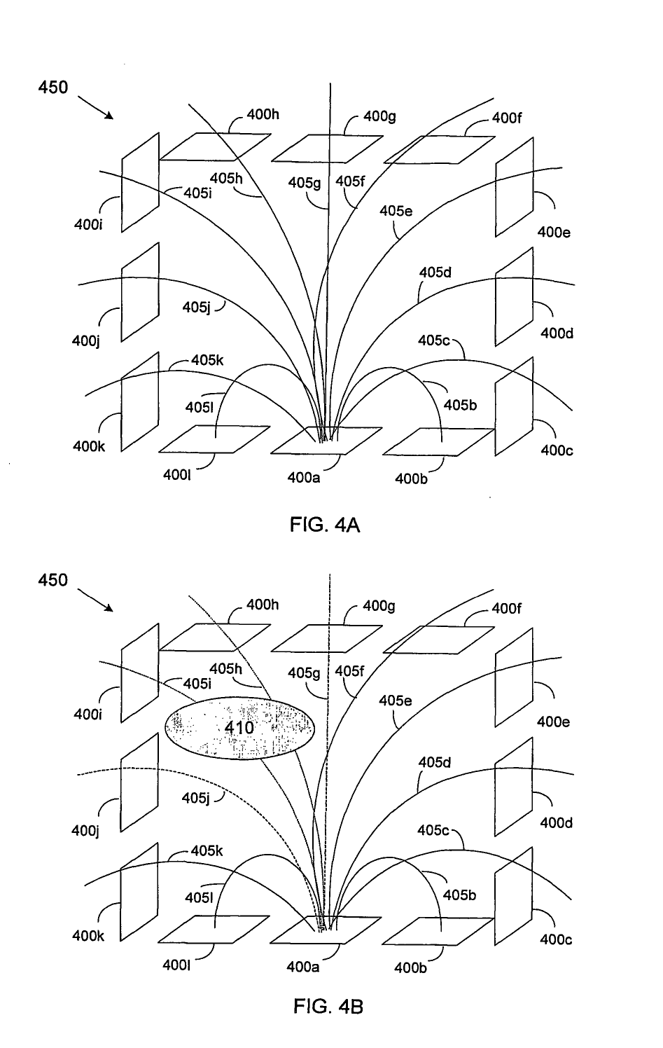

FIGS. 4A and 4B illustrate principles by which an array of coils may be employed to obtain spatial information about the dielectric properties of a body. Array 450 includes a plurality of coils 400a-400l. In FIG. 4A, some of the magnetic field lines generated from coil 400a when the coil is operated are shown as magnetic field lines 405b-405l. The partial magnetic field illustrated is merely schematic and represents a subset of the magnetic field lines that may be generated by coil 400a.

The exemplary magnetic field lines shown are employed to illustrate that, when coil 400a is operated, each of the other coils may experience a non-zero magnetic flux as a result of the magnetic field generated by coil 400a. The magnetic flux through each coil may induce a current in the respective coil proportional to the magnitude (i.e., the rate of change) of the flux. Accordingly, this induced current may be measured. While only magnetic field lines for coil 400a are illustrated, it should be appreciated that each of the coils, when operated, will generate a magnetic field that will induce a current in other coils in proportion to the magnetic flux passing through the coil.

In FIG. 4B, a dielectric body 410 is positioned within the array. The dielectric properties of the body 410 may affect the magnetic flux passing through one or more of the coils as a result of the operation of coil 400a. For example, the conductivity of body 410 in the presence of the magnetic field generated by coil 400a may induce eddy currents that oppose the magnetic field applied by coil 400a, affecting the magnitude of the flux through one or more of the coils. The change in magnetic flux, in turn, affects the current induced in the respective coil.

For example, magnetic field lines 405h and 405i may be substantially altered by the presence of body 410. Similarly, proximate magnetic field lines 405g and 405j may also be affected by the dielectric properties of body 410. Other magnetic field lines generated by coil 400a may be less or even negligibly affected by the presence of body 410. Accordingly, which coils experience a change in magnetic flux and the extent of the change provides an indication of the location of the dielectric body 410 and its dielectric properties.

For each operating coil, a pattern of magnetic flux perturbation (which results in a modification in induced current) will result in each of the other coils as a function of the location and dielectric properties of body 410. Since the location of each coil and the baseline current response of each coil in the array may be known, the patterns of flux modification yields information about the spatial distribution of a body's dielectric properties.

Applicant has identified and appreciated that in addition to the magnetic induction component (i.e., the inductive coupling between coils), the electric fields of the coil array provide another mechanism for determining the spatial distribution of dielectric properties of a body. The electric field of a coil results in part from potential differences across inductors and capacitors and also from any time-varying magnetic fields of the coil. These electric fields cause a current in conducting bodies and displacement currents in insulating (but polarizable) bodies. Displacement currents may modify the reactance of the coil, while currents in conducting bodies may modify coil resistance. Accordingly, the interaction of a coil array also has an electric field component (e.g., resistive and capacitive coupling between coils) that may be exploited to determine the dielectric distribution of a body.

In MIT, a back-projection algorithm similar to techniques widely used in X-ray CT is employed to determine dielectric characteristics of a body. For example, a solenoid coil and a gradiometer may be arranged such that a body to be imaged is disposed between them. A mutual inductance between the solenoid coil and the gradiometer may be measured and the solenoid/gradiometer pair then shifted relative to the body and another measurement taken. This process may be repeated, for example, around a circumference of the body to provide a series of “projections” of inductive coupling between the solenoid/gradiometer pair. However, back-projection has been shown, in some cases, to yield quantitatively and qualitatively inaccurate results. In MIT, these errors may be exacerbated by relatively poor sensitivity to loading changes of non-resonant detectors.

While back-projection may also be used to in the framework of resonant coils according to various aspects of the present invention, it may have limitations in extracting spatial information about the distribution of dielectric properties of a body. Applicant has recognized and developed methods of determining the dielectric distribution of a body that incorporates the magnetic component and the electric component of an array of resonant coils. In particular, Applicant has developed methods incorporating effects of resistive coupling, capacitive coupling and inductive coupling between resonant coils in an array.

Applicant has recognized that disturbances in the electric and magnetic fields caused by loading a coil array may be quantified using Maxwell's equations. In particular, Maxwell's equations may be employed to compute an impedance matrix of the coil array. The term “impedance matrix” refers generally to any ordered, related or otherwise correlated set of impedance characteristics of one or more coils in the presence of one or more other coils and/or dielectric bodies. An impedance matrix may include any impedance characteristics, for example, S-parameters or other measurements from which impedance may be derived.

A dielectric body may have spatially varying electric conductivity, permittivity and/or permeability. For example, a body may have an electrical conductivity σ(\vec{x}), electrical permittivity ε(\vec{x}), and magnetic permeability μ(x) where the vector x is a direction of the one or more axes over which the conductivity and permittivity vary. Variations in magnetic permeability μ may be small in biological tissues, in which case, the permeability may be represented by a constant value μ₀. However, some materials, such as metals, may have appreciable and measurable variation in magnetic permeability.

It should be appreciated that electromagnetic field energy is reduced when work is done on matter within and under the influence of the field. For example, when current is induced in a dielectric body, the energy in the inducing electromagnetic field is reduced in an amount proportional to the energy of the induced current. Accordingly, conservation of energy principles may be employed to ascertain various characteristics of a dielectric body by measuring the energy in the EM fields “lost” to the dielectric body.

For example, consider an array of N coils arranged in a substantially known relationship to one another. Let \vec{E}ᵢ(\vec{x}) and \vec{B}ᵢ(\vec{x}) denote, respectively, electric and magnetic fields resulting from a unit current in the i^{th} coil of the array. If the currents and fields are time-varying (e.g., of the form e^{-iωr}) an impedance matrix for the array may be expressed as,

$${Z}_{ij} = {\int }_{V} \{ σ ( x ) {\overline{E}}_{i}^{*} ( x ) \cdot {\overline{E}}_{j} ( x ) - ⅈω [ ɛ ( x ) {\overline{E}}_{i}^{*} ( x ) \cdot {\overline{E}}_{j} ( x ) - {μ ( x )}^{- 1} {\overline{B}}_{i}^{*} ( x ) \cdot {\overline{B}}_{j} ( x ) ] \} + {\int }_{S} {\overline{E}}_{i} ( x ) \times {\overline{B}}_{j}^{*} ( x ) \cdot ⅆ \overline{S} \qquad ( 3 )$$

where Zᵢⱼ is the impedance of the i^{th} coil in response to operating the j^{th} coil. The first term of equation 3 gives the resistive loading of the array from ohmic losses and the second and third terms give the capacitive and inductive loading of the array, which modify the array's reactance. The last term incorporates the effects of energy radiated out of a volume of interest. Stated differently, equation 3 describes loading effects of a body by considering each of resistive, capacitive and inductive coupling between the coils in the array, expressed in terms of coil impedance.

The first integral in equation 3 may be evaluated for some suitably large volume surrounding the coil array. Radiation losses, which are represented by the final term in equation three, may be incorporated by integrating the Poynting vector (i.e., the cross product of the electric and magnetic fields) over a surface that describes a volume of interest. This volume of interest, referred to as the “imaging volume”, typically includes at least a portion of the loading body. The imaging volume, therefore, refers generally to the portion of space where it is desired to ascertain the dielectric distribution, for example, the distribution of conductivity and/or permittivity characteristics.

Equation 3 illustrates some of the spatial encoding mechanisms described above. For example, the first term of equation 3 (i.e., the resistance matrix) measures the overlap of the body's conductivity with a product of the electric fields of the i^{th} and j^{th} coil as a function of space. Spatial encoding by mutual inductance is implicit in equation 3. That is, the electrical and magnetic fields themselves depend on the spatial distribution of the dielectric properties of the body. For example, the “blocking” or “screening” of the magnetic field by eddy currents as described in connection with FIGS. 4A and 4B illustrate one way in which the electromagnetic fields depend on the dielectric properties of the body being imaged.

It should be appreciated that inductive coupling between coils is fully expressed in the final term. This is in contrast to the linear approximation and back-projection techniques used in MIT. In further contrast, equation 3 fully expresses the resistive and capacitive coupling between the coils, providing additional information from which determinations about the dielectric distribution within an imaging volume may be obtained. By considering both the electric and magnetic field components of a coil array, various aspects of the present invention may provide a full description of the relationships between coils in an array in the presence of a loading body.

Equation 3 expresses the impedance of a coil array in terms of the electromagnetic fields generated by the array and the dielectric distribution of the imaging volume (e.g. a volume including a loading body) based on conservation of energy principles. However, it should be appreciated that there may be other ways of expressing coil array impedance characteristics in terms of the dielectric properties of a body that are suitable for implementing various aspects of the present invention and other formulations may be used without departing from the scope of the invention.

For example, dielectric properties of a body may be determined by computing currents that result from applying a known set of voltages to an array of resonant coil (e.g., using a finite difference time domain (FDTD) algorithm as described in more detail below). For example, a matrix of induced current values Iᵢⱼ(t) may be computed, where i indicates the coil where a known voltage V is applied and j indicates the coil at which an induced current is being computed.

By using Ohm's law and by taking the Fourier transform of the current and voltage data, the ‘intrinsic’ impedance of the coils may be expressed as,
Z_{intrinisic}(ω)=V(ω)I⁻¹(ω), (4)

where V is a diagonal matrix containing the known voltages. The input impedance of the coil array may then be computed by combining Z_{intrinsic} with the impedance of the matching circuitry. For example, in coils similarly arranged as shown in FIG. 2, the parallel circuit computation may be expressed in matrix form as,
Zᵢₙₚᵤₜ=(Z_{match}⁻¹+Z_{intrinsic}⁻¹)⁻¹ (5)

However, other forms may be used as well and may depend on the arrangement and topology of the coils.

It should be appreciated that by solving Maxwell's equations within an imaging volume, not only may dielectric properties of a loading body be determined but other electromagnetic properties as well. For example, properties of the electric and/or magnetic fields may be determined as a function of space. Accordingly, an image may be formed having intensity values indicating a magnitude, a direction, and/or phase of the electromagnetic environment as a function of space within the imaging volume.

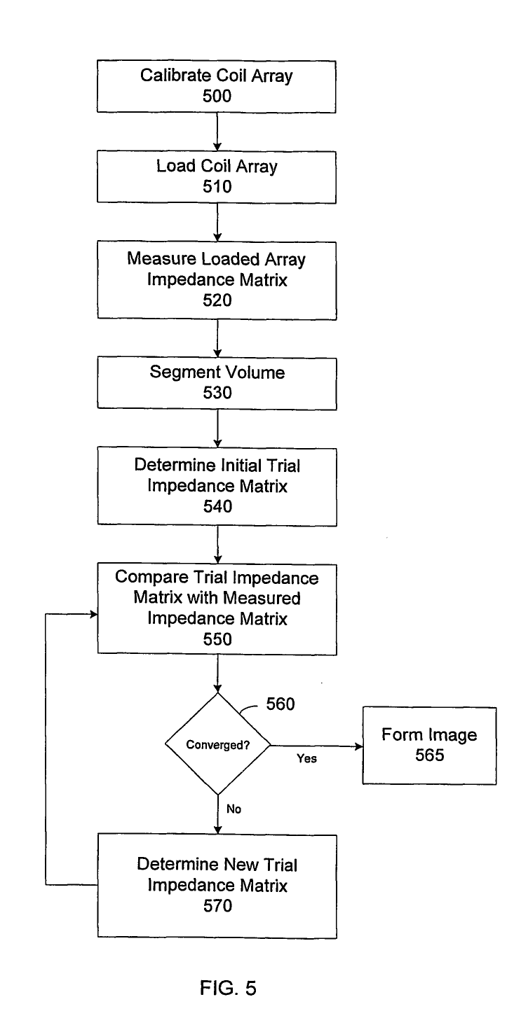

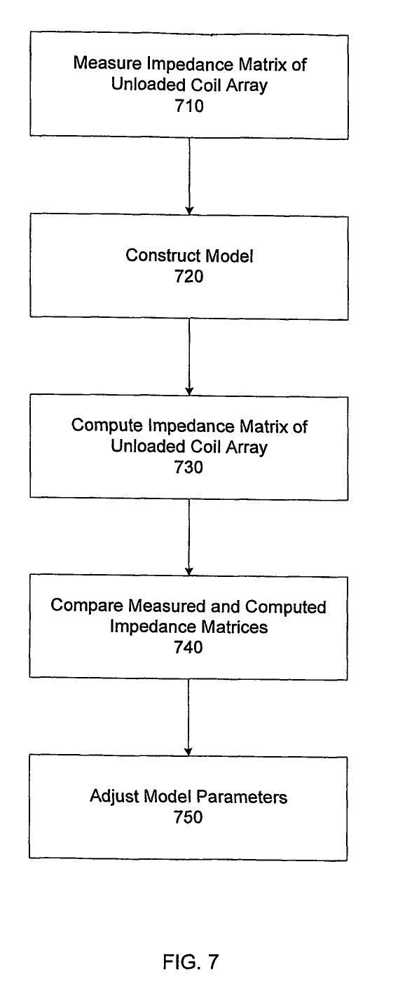

FIG. 5 illustrates one embodiment according to the present invention of a method for obtaining image information associated with the dielectric properties of a body from a coil array. In step 500, the coil array may be calibrated. Calibration may include various tuning and/or adjusting of parameters associated with the coil array as described in greater detail in connection with FIG. 7. Generally, calibration is performed in the absence of a load. However, in some embodiments, calibration may not be necessary or desirable and may therefore be omitted.

In step 510, the coil array may be loaded with the body to be imaged. For example, in FIG. 6, body 610 may be placed proximate coil array 600. In step 520, the impedance matrix of the coil array may be measured in the presence of the load at a number of frequencies. By operating the coils over a range of frequencies and measuring impedance characteristics, information about resonant frequency shifts and other changes in resonant properties may be incorporated. For example, an impedance matrix obtained as a function of frequency may incorporate resonant frequency information that may be used to determine the dielectric distribution of an imaging volume.

The impedance of each coil may be measured using any of various matching networks and/or network analyzers as described in more detail in connection with FIG. 9 or using any other suitable equipment and/or circuitry. The impedance matrix may be measured by operating each coil in isolation or by operating multiple coils in tandem and measuring induced currents, voltages, or other electrical properties in each of the coils. In a tandem operation, coils may be operated in pairs, in larger groups, or simultaneously as an entire array.

In step 530, an imaging volume, including at least a portion of the body, may be segmented into a plurality of regions. In general, an image is a set of intensities describing the properties of a corresponding region of the imaging volume. Accordingly, a volume containing a portion of the body to be imaged may be segmented into a plurality of regions, each region having an associated voxel (or pixel) in the resulting image that describes at least one intensity of the corresponding region.

For example, a conductivity image may include voxels representing the value of the conductivity of material located in the corresponding region of space. Similarly, a permittivity image may be produced, or an image having intensities that incorporate both the conductivity and permittivity value of the associated region of the imaging volume into an intensity value. Images may also be produced having intensities that represent a broad range of electromagnetic properties, including, but not limited to, permittivity, conductivity, permeability, electric field, magnetic field and/or any combination of the above.

The imaging volume may be segmented into regular or irregular regions. For example, in FIG. 9, the imaging volume is segmented into a regular 3D grid of equal sized and uniformly distributed cubes. However, one or more regions could be of different size or shape. For example, a priori information (e.g., information obtained from another imaging modality, separate measurement, knowledge of the body, etc.) may be used to segment the imaging volume intelligently to facilitate higher resolution images as discussed in greater detail below.

In step 540, an initial trial impedance matrix may be determined. In one embodiment, each voxel is assigned an initial trial conductivity and/or permittivity value, that is, each region has an associated trial value or values. The trial values may be assigned arbitrarily or they may be assigned according to a priori information about the body being imaged. For example, regions of suspected homogeneity may be identified from other imaging modalities such as an MRI image, or estimates of dielectric properties of one or more of the regions (e.g., from known characteristics of the body), may be obtained from a separate source, or obtained from computational models approximating the body.

It should be appreciated that in some environments (i.e., when certain types of bodies are being imaged), permeability may also be expected to vary as a function of space while in other bodies permeability may be assigned as the permeability of free space or some other constant value. Accordingly, some embodiments include assigning trial values of permeability to the imaging volume. The trial values assigned to the imaging volume may then be used to compute an initial trial impedance matrix.

For example, the trial conductivity and permittivity values (and in some instances trial permeability values) along with the electromagnetic fields computed using Maxwell's equations may be employed in the relationship expressed in equation 3 or to compute current amplitudes in each of the coils resulting from known applied voltages in order to compute impedance characteristics of the coil array. It should be appreciated that any expression describing impedance characteristics as a function of the dielectric properties of the body and the electromagnetic fields of the array may be used to determine an initial trial impedance matrix.

In step 550, the initial trial impedance matrix may be compared to the measured impedance matrix to determine if the trial values (i.e., the dielectric distribution) assigned to the body to form the trial impedance matrix are a satisfactory description of the conductivity and/or permittivity distribution of the body as indicated by the measured impedance matrix. That is, the trial impedance matrix and the measured impedance matrix may be compared to determine if they are sufficiently similar, or that a distance (e.g., a comparison metric) between the two is sufficiently small. In one embodiment, the measure of similarity between the trial and measured impedance matrix may be a least squares distance, although any measurement that indicates similarity between the trial and measured impedance matrices may be suitable.

In step 560, a distance between the trial and measured impedance matrix is evaluated. If the two matrices are determined to be close enough, that is, if the trial impedance matrix has converged to substantially the measured impedance matrix, or the distance between them is acceptably small, an image may be formed from the conductivity and/or permittivity values used in the formation of the trial impedance matrix. Otherwise, the trial impedance matrix may be updated.

In step 570, when it is determined that the trial impedance matrix and the measured impedance matrix are too dissimilar (i.e., a distance between the two matrices is too great) the conductivity and/or permittivity values assigned to one or more voxels may be adjusted such that an updated trial impedance matrix formed from updated trial values is nearer the measured impedance matrix than during the previous iteration.

Any of various methods for iteratively adjusting the dielectric properties assigned to each voxel including, but not limited to, gradient descent, searching algorithms such as simulated annealing, statistical methods such as expectation maximization, or any other optimization method of solving a set of linear or non-linear equations, etc., may be used to converge the trial impedance matrix to the measured impedance matrix. Steps 550-570 may be repeated until the trial impedance matrix has converged or is determined to be satisfactorily close to the measured impedance matrix. In addition, analytical or other non-iterative methods may be used for finding a solution.

In step 565, when the distance between the trial impedance matrix and the measured impedance matrix has converged to a local minimum, an image may be formed from the trial values. For example, an image having a plurality of voxels may be formed wherein each voxel is assigned an intensity value related to the conductivity and/or permittivity value of an associated region of the imaging volume.

It should be appreciated that the method described in connection with FIG. 5 depends in part on computing an impedance matrix given a set of conductivity and permittivity values. Accordingly, results may depend on how accurately the impedance matrix may be computed. Applicant has developed methods for calibrating a coil array such that computed impedance matrices of the unloaded array agree with measured impedance matrices of the unloaded array. A calibrated coil array may give a baseline indication of the accuracy of impedance matrix computations.

FIG. 7 illustrates one embodiment of calibrating an array of coils according to the present invention. For example, the calibration method described below may be used in step 500 in FIG. 5 to improve the accuracy of impedance matrix computations. Computing an impedance matrix may include building a model of a coil array and an imaging volume.

The term “model” refers generally to a description or representation of one or more objects (e.g., a coil array, an imaging volume and its contents, such as a body to be imaged, etc.). While a model may emulate a real object (e.g., a model of coil array may have a real coil array counterpart), a model is virtual, typically embodied by one or more mathematical descriptions. For example, a model may include descriptions of the geometry of the one or more objects, parameters that describe the characteristics of the object, etc. Models may be stored electronically, for example, on computer memory or as part of a executable program stored on computer memory.

In order that a model emulate at least some properties of the real object, one or more functions of the object being modeled may be simulated using the model representation. Simulation refers to computing a function, operation or action such that the model representation behaves similarly to its real counterpart.

Consider the case when the real objects being modeled include a coil array. When one or more coils in the array are operated, electromagnetic fields are generated. This electromagnetic environment in turn affects properties of the coil. Simulation of a coil array model may include computing the electromagnetic environment of the coil array either in the presence or absence of a body acting as a load. Methods and algorithms for performing a simulation are often embodied in one or more software programs operating on the model or representation of the system being simulated.

For example, a model of a coil array may be simulated by solving Maxwell's equations within an imaging volume defined as part of the model. By solving Maxwell's equations during simulation, the electromagnetic environment of an imaging volume may be determined such that, together with trial conductivity and permittivity values (during calibration, trial values may be set to the conductivity and permittivity values of air), may be used in the expression of equation 3 to compute an impedance matrix.

One method of simulating a model of a coil array (both with and without a load) includes solving Maxwell's equations according to a Finite Difference Time Domain (FDTD) algorithm. In FDTD, a volume of space (e.g., the imaging volume) with or without one or more objects (e.g., a body to be imaged) is partitioned into a lattice or mesh. The electromagnetic fields in each region of the mesh are solved for according to an applied set of initial conditions (e.g., conductivity and/or permittivity distribution, coil geometry, etc.) and boundary conditions (e.g., the extent of the imaging volume, etc.).

While FDTD may provide a fast and effective method for simulating the operation of a coil array and computing an impedance matrix, any of various other computational methods including Chebyshev polynomial expansion, finite element, finite difference frequency domain (FDFD) algorithms, any of various other frequency domain computational methods etc., may be suitable for simulation and are considered to be within the scope of the invention.

In step 710, an impedance matrix of the unloaded array may be measured. Various methods of measuring the impedance matrix may be suitable to obtain a measurement with which to calibrate a model of the unloaded array. In one embodiment, each coil may be operated in isolation and the current induced in each of the other coils measured. For example, a pulse from an RF power source, or any other electrical stimuli (e.g., a voltage, a current, etc.) may be applied at the input of one of the coils and the currents in each of the other coils measured. For example, the power source may act as a voltage or current source providing a broadband electric field pulse. The pulse may be, for example, a “chirp” containing a range of frequencies. The range of frequencies may include the nominal resonant frequency of one or more of the coils and chosen to include an approximate shift in resonant frequency. This process may be repeated at the input of each of the coils in the array.

In step 720, a model of the unloaded array may be computed. The model may be computed at any time and portions may only need to be computed once for a given coil array. For example, parameters of the coil array that do not change from one calibration to the next (e.g., number and arrangement of coils, various coil properties such as conductivity and capacitor values, etc.) may be generated and stored. Parameters such as the size of the imaging volume, quantization size of the volume, etc., may need to be input into the model each time the imaging environment changes as described in further detail below.

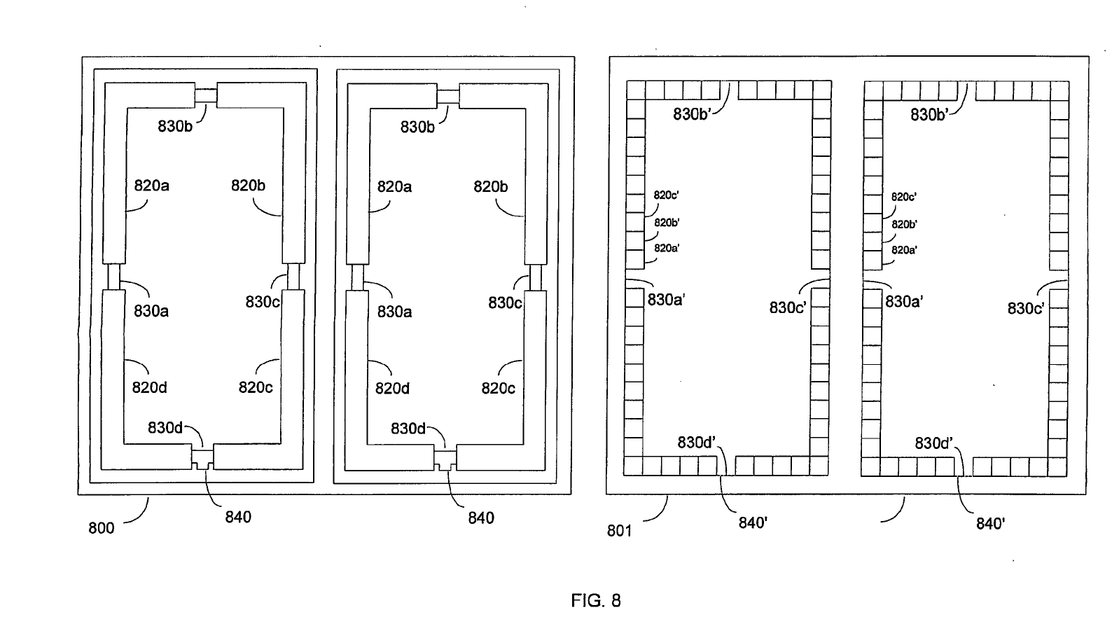

The array coils and the imaging volume may be partitioned into a lattice, that is, the imaging volume may be segmented into a plurality of regions. FIG. 8 illustrates a two coil array 800 and a corresponding model of the array 800′ shown as a collection of discrete squares. The conductive strips 820a-82d may be simulated as a plurality of discrete units (e.g., rectangles 820a′, 820b′, 820c′, etc.) having electrical characteristics of a high conductivity material. Likewise, capacitors may be simulated as high permittivity insulating materials illustrated by simulated capacitors 830a′-830d′. Each coil may have a simulated port 840′. It should be appreciated that other parameters and properties of the array may be defined in the model in order for the model to be simulated as desired.

The imaging volume may be defined as any suitable volume proximate the coil array. The imaging volume may then be partitioned into discrete regions. It should be appreciated that the imaging volume can be partitioned at any desirable resolution. Other parameters of the imaging environment may be defined and added to the model.

In step 730, an impedance matrix of the unloaded array may be computed by simulating the operation of the coil array. For example, FDTD simulations may be performed on the model to compute impedance characteristics of each coil in the absence of a load. In step 740, the computed impedance matrix may be compared to the measured impedance matrix to determine if the model generates values that generally agree with measured values.

In step 750, parameters of the model may be adjusted to compensate for differences in the computed and measured impedance matrices. Parameters of the model that may facilitate calibrating the model include, but are not limited to, additive correction to the array's inductance and/or resistance matrix, adjustments to the nominal value of the matching capacitors (e.g., to account for errors in capacitor fabrication and/or stray or parasitic capacitance in the matching circuitry), etc., may be adjusted so that the computed impedance matrix agrees with the measured impedance matrix as discussed in further detail below. Accordingly, a coil array model may be calibrated such that before the array is loaded, impedance matrix computations can be made to substantially agree with measured impedance matrices of the unloaded array.

As described above, each region of the body being imaged may have a corresponding voxel describing one or more dielectric properties of the associated region. The smaller the subdivisions of the imaging volume (i.e., the smaller the regions are chosen) the greater the resolution of the resulting image. However, the size of the regions may be limited by the resolving power of the coil array. That is, the resolution may depend in part on the coil array's ability to ascribe loading effects on the properties of the array to individual regions of the imaging volume, which depends in part on the number and arrangement of coils in the array.

The resolution of an RFIM image may depend in part on the number of correlated or independent measurements that may be made of a coil array. For example, the size of an impedance matrix may be related to the resolution of the resulting image. This is, in turn, may be related to the number of coils in the array. As the number of coils in an array increases and/or their size decreases, a size of a region of an imaging volume of which the coil array can resolve a conductivity or permittivity value decreases. As such, the size of each region of an imaging volume is inversely proportion to the resolution of the image.

It should be appreciated that an impedance matrix may include both a resistance matrix, which encodes primarily conductivity distribution, and a reactance matrix, which encodes both conductivity (e.g., via eddy currents) and permittivity (e.g., via capacitive loading). Each of these matrices may provide N(N+1)/2 equations, where N is the number of coils. Considering redundant information, it is estimated that N²/2 voxels may be imaged with N coils. However, conditioning information, such as information obtained from MRI, other measurements, and/or any a priori information about an object to be imaged may greatly improve this number.

FIG. 9 illustrates one embodiment according to the present invention of an image acquisition system for obtaining an RFIM image of a body. Image acquisition system 900 includes a coil array 910 having a plurality of RF coils (e.g., coil 910a and 910b). Coil array 910 is illustrated as having 8 individual coils. However, the arrangement and number of coils illustrated in FIG. 9 is merely exemplary and is not limiting, as a coil array may be chosen to have any number of coils in any arrangement. For example, a coil array may be arranged such that coils are positioned on all sides or a desired number of sides of an object to be imaged instead of just underneath a body 950 as shown in FIG. 9.

Power source 915 may be coupled to coil array 910 to provide power to operate the coils. While power source 915 is shown schematically as connected generally to coil array 910, it should be appreciated that the power source may be connected to the coil array such that power (e.g. a voltage or current waveform) may be individually provided to each of the coils in the array. That is, each coil may have a separate port with which to receive power.

Coil array 910 may also include various blocking networks that are capable of selectively blocking current flowing around a given coil. Such blocking networks may effectively turn one or more coils off, for example, during calibration or during measurement of other coils in the array, etc. One or more baluns may also be included to prevent unshielded currents from flowing on the cables that connect the array to the other equipment in the system. Other circuitry may be included such that each of the coils in the array can be selectively operated.

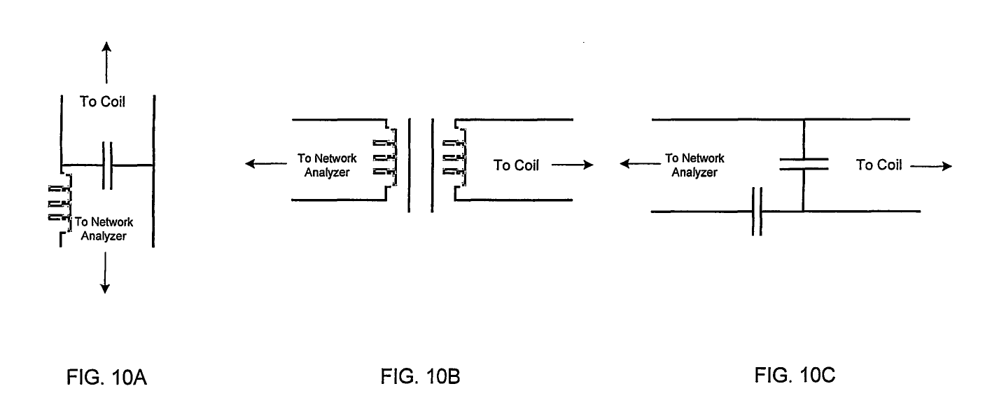

Matching circuitry 925 may also be coupled to coil array 910. As with the power source, matching circuitry is illustrated as generally coupled to the coil array. However, it should be appreciated that each coil may have its own matching circuitry such that various properties of the coil may be measured by measurement equipment 935. For example, a number of exemplary and suitable matching circuits are illustrated in FIGS. 10a, 10b and 10c. Any of various other matching circuits may be used such that properties of the coil array may be measured.

Measurement equipment 935 may be coupled to one or more of the matching circuits to measure properties of the coil array such as a voltage, current and/or impedance of individual coils in the array. Measurement equipment 935 may, for example, include a network analyzer having one or more ports connected to the matching circuitry 925 and capable of obtaining measurements of one or more properties of the coil array.

A computer 945 may be coupled to the measurement equipment 935 to receive the measurement of one or more properties of the coil array. Computer 945 may be any component capable of performing mathematic computations and/or logic operations. Computer 945 may be, for example, one or more microprocessors or digital signal processors. Computer 945 may also include a computer readable medium such as a memory capable of being encoded with instructions, for example, a program configured to perform various functions and operations when executed by one or more processors. Computer 945 may be included as part of the measurement device 935, for example, a processor included in the network analyzer or may be a separate component.

Computer 945 may be configured to perform computations to facilitate any of calibrating the coil array, modeling the coil array, computing impedances matrices, forming an image of dielectric properties of a body, etc. Computer 945 may also be coupled to a display 955 capable of rendering RFIM images acquired from measurements of the coil array.

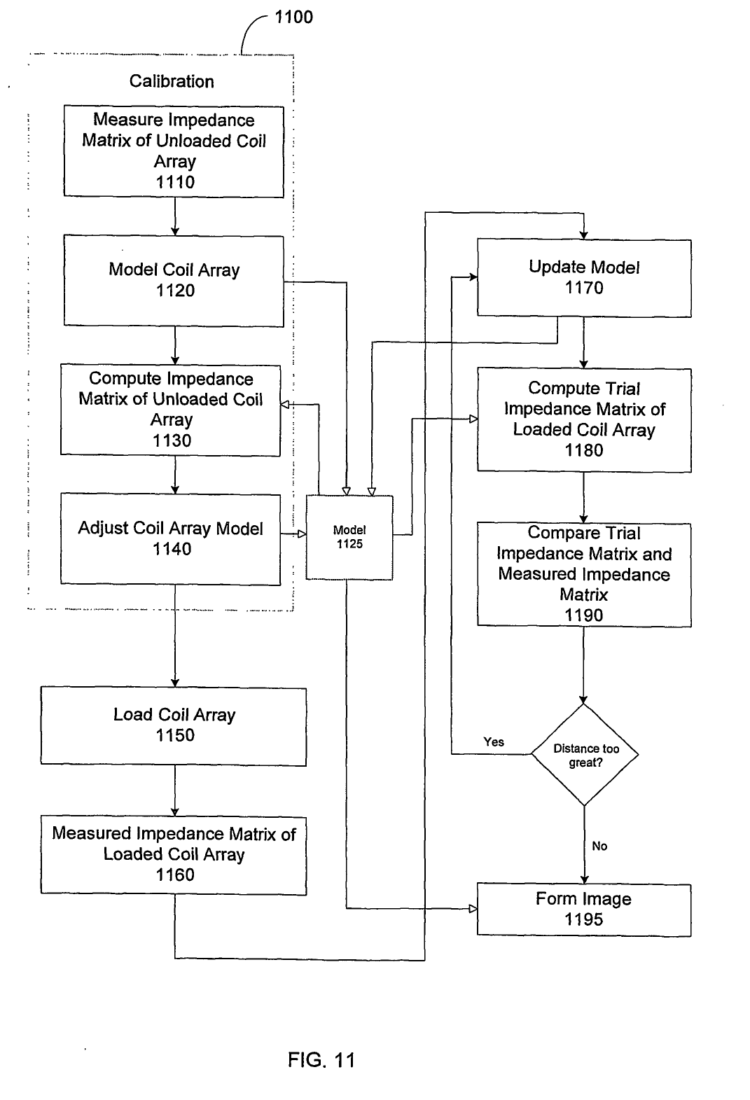

FIG. 11 illustrates one embodiment according to the present invention of forming an RFIM image of a body using an image acquisition system, for example, image acquisition system 900 illustrated in FIG. 9. In step 1100, the coil array may be calibrated. During calibration, power source 915 in FIG. 9, for example, may apply a waveform at a desired frequency (e.g., an RF pulse) to the coil array such that a current is generated in one of the coils. As discussed above, an impedance matrix may be formed from S-parameters measured by, for example, a network analyzer.

S-parameters describe the transmission and reflection of traveling waves, that is, they may represent the reflection and transmission coefficients between incident and reflection waves and are often measured as a function of frequency. The S-parameters may be used to describe the behavior and characteristics of circuit or network (e.g., a resonant coil). There exists a one to one correspondence between S-parameters and impedance and the two measurements can easily be converted into one another, for example, by S=(Z−Z₀)(Z+Z₀)⁻¹, where Z₀ is the match impedance of the RF source (e.g., 50 or 75 ohm BNC impedance).

Accordingly, a measured impedance matrix may be obtained by successively operating each coil at a range of different frequencies and measuring the S-parameters of the coil array. That is, power source 915, may supply power to each coil over a range of desired frequencies. Typically, the range of frequencies includes a nominal resonant frequency of the coil being operated. For example, a coil may be designed to resonate at a given frequency. As discussed above, when a coil is loaded, the resonant frequency may shift in response to the electromagnetic coupling between the coil and the load.

When a coil is operated at its resonant frequency, a maximum amount of energy is being coupled into the coil and a minimum amount of energy is being reflected back to the power source. This resonant condition manifests itself in a local minimum in the magnitude of the so-called S₁₁ parameter. Accordingly, if a given coil is operated at a range of frequencies including its nominal resonant frequency and the S₁₁ parameter measured at each desired frequency in the range, a plot of the S₁₁ parameters will show a dip in magnitude at the resonant frequency. When a coil is loaded and the measurements repeated, the dip in magnitude will have shifted due to the shift in resonant frequency of the coil. As such, resonant frequency shifts may be encoded in S-parameter measurements. In addition, changes in depth and breadth (in frequency) of this dip encode further information about the resonance.

In addition to the resonant frequency shift information encoded in the S-parameters, resistive, capacitive and inductive coupling between the coils may also be available from measurements of the S-parameters. For example, when a given coil is operated, some of the power is dissipated in the other coils. This power can be measured in the S-parameters of the other coils. The notation Sᵢⱼ will be used to indicate the S-parameter measured in the j^{th} coil in response to operating the i^{th} coil.

Accordingly, an impedance matrix may be obtained as Sᵢⱼ(ω), where ω are the frequencies over which the coil is operated and S-parameters of the array are measured. This measured impedance matrix may incorporate information about resonant frequency perturbation, resistive, capacitive and inductive coupling between the various coils in the array. This information may be employed to determined the dielectric properties of the loading body.

In step 1120, a model 1125 of the coil array may be constructed. In particular, a model may be defined such that the electromagnetic environment of the coil array resulting from operating an arbitrary number and arrangement of coils may be simulated. The geometry, properties and characteristics of the coil array may be described (e.g., as a mathematical representation) so that the operation of the coil array may be simulated. For example, an operator may program a model of the coil array by describing various operating parameters, values of virtual components (e.g., capacitor values, etc.), geometry of the array (e.g., number of coils and spatial relationship between coils), etc., and store the model on a computer readable medium, such as a memory stored, for example, on computer 945 in FIG. 9.

In addition, an imaging volume wherein a dielectric distribution will be computed may be defined. For example, in FIG. 9, imaging volume 905 may be defined and included in the model. For example, imaging volume 905 may be chosen to at least enclose a portion of a body that is desired to be imaged. The imaging volume may be partitioned into a plurality of regions (e.g., such as region 905a, 905b, 905c, etc).

It should be appreciated that the imaging volume may be partitioned in any number of ways and the volume illustrated is merely exemplary. For example, the size of each region may be chosen to provide a desired resolution of the resulting image. In addition, the regions may be chosen to be of different size and shape as suits a particular implementation (and in consideration of the body being imaged). The imaging volume may be chosen to enclose certain portions of the object, may be chosen to enclose the entire body, or chosen to enclose the body and the coil array and/or any peripheral space around the coil array that facilitates simulation of the coil array. The constructed model 1125 may be stored and later accessed during coil array simulation.

In step 1130, a computed impedance matrix may be generated. During the computation of a computed impedance matrix for calibration, each of the plurality of regions in the imaging volume may be assigned values corresponding to the dielectric properties of air to simulate an unloaded coil array. For example, conductivity values σ₀ and ε₀ may be assigned to the plurality of regions, where σ₀ and ε₀ are the conductivity and permittivity values of the empty imaging volume (e.g., air).

The model of the unloaded coil array together with the assigned dielectric properties of the empty imaging volume may be simulated, for example, by performing an FDTD simulation of the model. It should be appreciated that the model, the imaging volume, and the FDTD algorithm (in addition to other functionality) may be stored on a memory of computer 945, for example, as one or more software programs.

The simulation may compute an impedance matrix by determining the unloaded EM environment and, together with the assigned dielectric values, employ the relationship expressed in equation 3. In particular, since the conductivity and permittivity values have been assigned as a discrete function of space (i.e., the dielectric properties assigned to each region of the imaging volume), and the electric and magnetic fields generated by the coil array at various input frequencies may be calculated throughout the imaging volume (e.g., by solving Maxwell's equations during simulation), the impedance values of the coils may be computed.

Radiation losses may also be included in the simulation (even though the effects may be relatively small). In one embodiment, computation of radiation losses may be achieved in an FDTD simulation by choosing the appropriate boundary conditions. For example, the appropriate ‘out-going wave’ boundary conditions may be applied at the surface extent of the imaging volume. Accordingly, a computed impedance matrix and a measured impedance matrix may be obtained for the unloaded coil array.

In step 1140, this information may be used to adjust parameters of the model such that computation of impedance characteristics agree with measurement of impedance characteristics. Stated differently, the model may be adjusted so that simulation of the coil array comports with the actual operation of the coil array.

For example, additive correction may be made to the array's resistive, capacitive and/or inductive matrix. In some embodiments, this correction may mitigate errors that may arise due to the discrete nature of the simulation of the model. For example, in an FDTD simulation, the partitioned regions of the imaging volumes may not take into account, for example, the contribution to an array's impedance at distances very near the coils (i.e., at distances smaller than the quantization of the imaging volume). In addition, defects or imperfections in the fabrication of the coils may need to be compensated for by adjusting the parameters of the model. Errors in capacitor fabrication and stray capacitance in, for example, the matching circuitry may be accounted for as well. Other adjustments may be made as well to compensate for differences in the model and the real coil array being represented by the model.

When a network analyzer is calibrated, the length of a cable connecting the network analyzer to the coil array may be taken into consideration. However, small deviations from the assumed length of the cable (e.g., due to small additional circuitry in and near the connector, such as a BNC connector) may add unaccounted for phase to the measured S-parameters, which may cause the computed S-parameters and measured S-parameters to disagree (i.e., phase shifted from one another as a function of the cable length deviation at each coil port). This phase may be accounted for in the model by shifting the phase of the measured or computed S-parameters accordingly. For example, phase correction may take the form:
Sᵢⱼ(ω)→e^{i(φ}^{ᵢ}^{(ω)+φ}^{ᵢ}^{(ω))}Sᵢⱼ(ω) (6)

where φ_{i(ω)} is the phase acquired at coil port i at frequency ω (such phases increase linearly with frequency). The various adjusted parameters of the model may then be used to update model 1125.

In step 1150, the coil array may be loaded with a body to be imaged. For, example, body 950 in FIG. 9 may be placed proximate the coil array 900 such that the loading effect of the body can be measured, for example, in the loaded S-parameters of the coil array.

In step 1160, a measured impedance matrix for the loaded array may be measured. Measuring the impedance matrix with the body proximate the coil array may be performed in a manner similar to that described in connection with measuring the impedance matrix of the unloaded array. For example, in the system of FIG. 9, each coil may be operated successively over a range of frequencies. At each frequency, the S-parameters for the coil array may be obtained via matching circuitry 925 and measurement device 935.

In step 1170, the model may be updated according to an initial approximation of the dielectric distribution of the imaging volume with the addition of the loading body. For example, the imaging volume may be assigned initial trial conductivity and/or permittivity values. The trial values may be assigned arbitrarily or may be assigned according to a priori information about the imaging body. Any a priori information available that may be employed to condition the simulation may facilitate faster convergence times and may reduce opportunities for the simulation to converge to an undesirable local minimum, and may result in the ability to obtain higher resolution images.

In step 1180, operation of the coil array may be simulated according to the model 1125 to produce a trial impedance matrix. Simulation may be performed substantially as described above for calibration using the updated dielectric properties of the imaging volume. In step 1190, the trial impedance matrix may be compared with the measured impedance matrix to determine a distance between the two matrices. This comparison may provide an indication of how closely the trial values approximate the actual distribution of the dielectric properties of the imaging volume.

If the distance is determined to be too great, that is, if the distance suggests that the distribution of dielectric properties is not a close enough approximation of the true dielectric distribution of the body, the trial values of conductivity and/or permittivity may be updated. For example, the trial values may be updated according to a method of gradient descent. According to one method, the system of equations expressed as,
Zᵢⱼ^{trial}(ω,εₙ,σₙ)=Zᵢⱼ^{meas}(ω), (7)

may be solved, where (ij) indicates the coil pair index and the index n enumerates the regions into which the imaging volume is divided. Solving the system of equations expressed in equation 7 may be achieved in a number of ways. In one embodiment, the Port optimization library developed by AT&T is used, although various other optimization schemes may be employed.

As is often the case with optimization schemes, the inquiry of whether a problem is well-posed of often addressed, for example, whether the system of equations expressed in equation 7 is well-posed. This inquiry typically involves asking whether a solution exists, and if so, if the solution is unique. In the RFIM framework as described above, these questions may be answered by considering the so-called “electrical prospection” problem.

Consider an imaging volume V. By specifying a tangential component E_{T} of the electric field on the boundary of V, Maxwell's equations may be used to compute the electric and magnetic fields throughout the entirety of V. In particular, the normal component B_{N} of the magnetic field on the boundary of V may be computed. This constitutes a map of the form:
B_{N}=F(E_{T},ε,σ) (8)

for each point on the boundary of V. In RFIM, E_{T} may be controlled by means of the coils. For instance, consider an imaging volume whose surface is uniformly tiled by a very large number of rectangular coils. By applying known voltages at the input port of each coil, tangential electric fields of known magnitude may be generated. The normal component of the magnetic field B_{N} may then be obtained by measuring the current induced in each of the coils via Faraday's law of induction. By using an array that consists of an infinite number of infinitely small coil elements and that fully encloses the volume V, the function F in equation 8 may be fully determined. It has been shown that the measurement of this function is sufficient to uniquely determine local conductivity and permittivity inside V.

Since, in practice, only a finite number of coils are available with which to test the function F appearing in equation 8, there may be pathological instances where the inverse problem is ill-posed. However, such pathological cases are common to all techniques of medical imaging and remote sensing in general. In practice, these ill-posed cases result in image artifacts that a trained practitioner can recognize and correct and/or that can be avoided by appropriate array design and/or conditioning the reconstruction of the image.

Steps 1170-1190 may be repeated until it is determined that the trial dielectric distribution is a satisfactory approximation of the dielectric distribution within the imaging volume, and hence, the dielectric properties of at least a portion of the body being imaged. That is, the distance between a final trial impedance matrix and the measured impedance matrix is sufficiently small (or it has been determined that the algorithm has converged or both).

In step 1195, the conductivity and/or permittivity values assigned to the image volume to compute the final trial impedance matrix may be used to form an RFIM image. For example, each voxel in the image may have an intensity related to a conductivity and/or permittivity value assigned to an associated region of the imaging volume. A conductivity and a permittivity image may be formed, or a single image may be formed deriving intensity values from some combination of the conductivity and permittivity values. The resulting RFIM image may be stored in memory or displayed, for example, on display 955 as shown in FIG. 9.

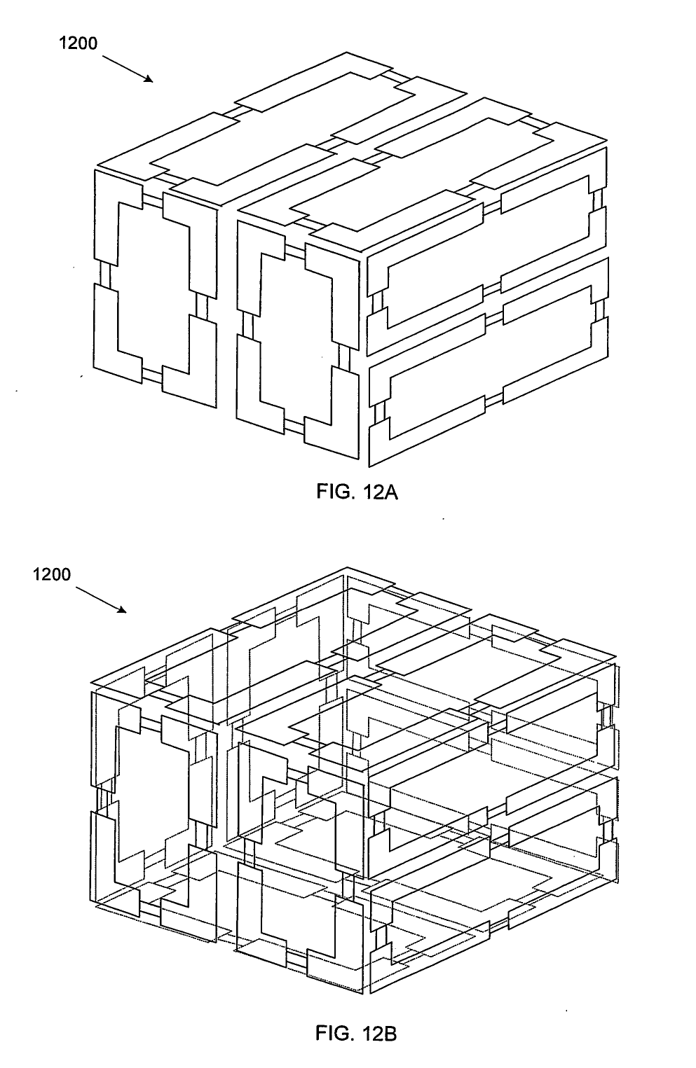

It should be appreciated that a coil array may comprise any number of coils in any arrangement. For example, FIGS. 12A and 12B illustrate one embodiment according to the present invention of a coil array arrangement having a plurality of coils arranged substantially in a rectangle configuration. FIG. 12B illustrates the same array as in FIG. 12A, however, all of the coils in the array are shown using dotted lines for coils on the hidden faces.

In coil array 1200, opposing faces of the rectangular array have coils that may be positioned essentially orthogonal to one another. In particular, generally rectangular coils on opposing faces may have the axis along their larger dimension positioned essentially perpendicular to one another. In other embodiments, coils on opposing faces are arranged essentially parallel to one another. Other non-parallel or non-orthogonal arrangements may be suitable to form a generally closed coil array. Coil array 1200 may, for example, define the outer boundary of an imaging volume or the imaging volume may be chosen to enclose the coil array.

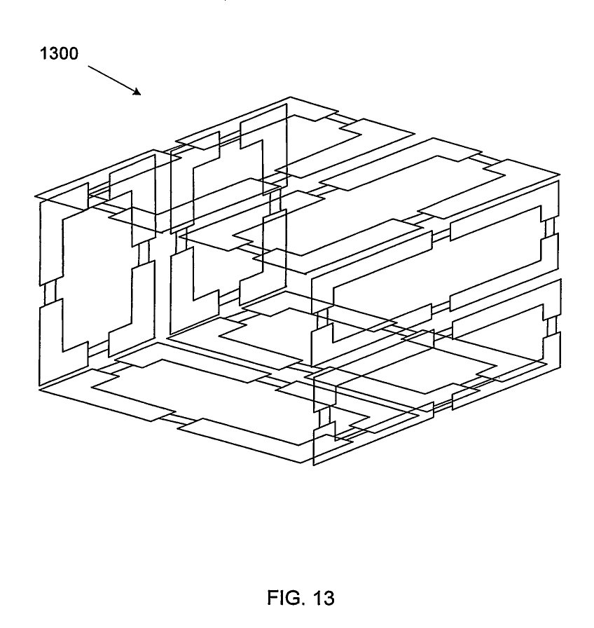

FIG. 13 illustrates another embodiment of a coil array according to the present invention. Coil array 1300 may be substantially similar to coil array 1200. However, coils on two of the faces may be removed such that the coil array has an opening. Coil array 13 may be well suited for imaging the torso of a patient or portions of an object that may not fit entirely within a closed array.

Coils in an array may be arranged in any other arrangement such as in a generally cylindrical configuration around a body to be imaged or any other layout wherein loading effects of a body may be measured. By increasing the number of coils in the array, the resolution of an RFIM image may be increased. In addition to the number of coils, the arrangement of coils may affect the resolution of image, in part, due to conditioning of the imaging environment.

In one embodiment according to the present invention, a coil array layout may be chosen in order to increase the amount of information that may be encoded about a loading body. In some circumstances, information about a body to be imaged may be available. For example, in a human body, approximate size and shape of the body may be available. In addition, nominal values for conductivity and permittivity may be available. This information may be used to evaluate the conditioning of a particular array layout.

In some embodiments discussed above, a system of equations may be solved in order to determine the conductivity and permittivity distribution within an imaging volume. For example, the system of equations expressed in equation 7 may be solved. This system of equations is generally a non-linear system of equations. However, the system of equations may be approximated by a linear system of equations such that linear techniques may be used to determine the conditioning of the system of equations.

For example, a measure of how well conditioned a system of linear equations is may be determined from the condition number. Consider a system of linear equations expressed as Ax=b, where it is desired to solve for x in terms of b with a coefficient matrix A. When the coefficient matrix A has a singular value of zero, x cannot generally be solved for because A⁻¹ does not exist. In the numerical framework, singular values close to zero may prevent solving for x in a similar manner. Arrangements in which the array layout is not well conditioned lead to these degenerate or near degenerate conditions.

In order to avoid such coil layouts, an array may be arranged such that the coefficient matrix (i.e., a system of equations) obtained from, for example, simulating the coil array with approximations to the make-up of the body (e.g., size, shape and dielectric distribution) does not have zero or near zero singular values. One measure may include computing a ratio of a largest singular value to a smallest singular value. When the smallest singular value is small compared to the largest singular value (i.e., when the ratio is large) a coil layout may not be well conditioned and may need to be adjusted. Accordingly, different coil layouts may be arranged such that layout is suitably conditioned.

In one embodiment, the Jacobian of the non-linear system of equations is used as a linear approximation of the non-linear system of equations. The singular values of the Jacobian matrix may then be determined analytically or by any of various methods such as the Jacobi method, Gauss-Seidel, Singular Value Decomposition (SVD), or by any other method suitable for obtaining the singular values of the Jacobian matrix. The singular values may be compared in any number of ways to determine whether the layout is well conditioned. This process may be repeated until a layout with suitable conditioning is obtained.

In another embodiment according to the present invention, a single coil may be used to image a loading body. For example, the spatial relationship between the single coil and the body may be varied and measurements of the loading effect taken at each placement. It should be appreciated from the foregoing that the S₁₁ parameter measures the reflected energy in a coil resulting from operating the same coil. Accordingly, this information may be available absent interactions with any other resonant coils to determine the dielectric distribution of the body.

Furthermore, one or more coils may be added to increase the amount of information available (e.g., resistive, capacitive, and/or inductive coupling between coils) for determining the dielectric properties of the body. These additional one or more coils may be varied relative to the body such that multiple measurements may be taken. By varying the spatial relationship between one or more coils and the loading body in relatively small increments, higher resolution images may be achieved without requiring relatively large numbers of coils to be employed in an array.

Employing resonant coils (as opposed to inductive coils such as solenoid coils) facilitates using the sensitive loading properties of resonant coils to determine the dielectric distribution of a volume of space. In addition, by considering a fuller electromagnetic environment of the volume (e.g., resistive, capacitive and/or inductive coupling), more information is available that may result in higher quality, better resolution images.

It should be appreciated that various embodiments according to the present invention result in relatively inexpensive and portable equipment. While MRI coils may be used to practice aspects of the present invention, simpler and less expensive resonant coils may also be appropriate. For example, various equipment symmetries, tuning and detuning circuitry, gradient coils, etc., needed for MRI are not required in RFIM.

In addition, expensive and bulky magnets required for MRI imaging may be eliminated. Accordingly, it may be relatively inexpensive to produce an RFIM imaging system. Moreover, the equipment has a relatively small footprint and may be capable of being transported from one location to another, making it suitable for mobile and/or emergency situations.

It should be appreciated that various aspects of the present invention may be may be used alone, in combination, or in a variety of arrangements not specifically discussed in the embodiments described in the foregoing and is therefore not limited in its application to the details and arrangement of components set forth in the foregoing description or illustrated in the drawings. The invention is capable of other embodiments and of being practiced or of being carried out in various ways. In particular, various aspects of the present invention may be practiced with any number of coil types and arrangements. For example, generally planar coils, birdcage coils, surface and volume coils may be used alone or in any combination with the any of the various imaging techniques described herein.

In addition, various aspects of the invention described in one embodiment may be used in combination with other embodiments and is not limited by the arrangements and combinations of features specifically described herein. Various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

What is claimed is:

1. A method of determining one or more properties of a body positioned proximate an array of coils having one or more resonant properties, the method comprising acts of:
detecting a change in at least one resonant property of at least one of the coils in the array; and
determining at least one electromagnetic property of at least one region of the body from the change in the at least one resonant property.

2. The method of claim 1, wherein the act of detecting the change in at least one resonant property includes an act of detecting a change in at least one resonant frequency of at least one of the coils in the array.

3. The method of claim 1, wherein the act of determining the at least one electromagnetic property includes an act of determining at least one of a conductivity, a permittivity, and a permeability of the at least one region of the body.

4. The method of claim 1, wherein the act of determining the at least one electromagnetic property includes an act of determining at least one of a magnitude, a direction, and a phase of an electric field at the at least one region of the body.

5. The method of claim 1, wherein the act of determining the at least one electromagnetic property includes an act of determining at least one of a magnitude, a direction, and a phase of a magnetic field at the at least one region of the body.

6. The method of claim 1, further comprising an act of forming an image having a plurality of voxels, each voxel of the plurality of voxels having an intensity related to a respective one of the at least one electromagnetic property.

7. The method of claim 1, wherein the act of detecting a change in the at least one resonant property includes an act of measuring at least one property of the array of coils indicative of the change in the at least one resonant property.

8. The method of claim 7, wherein the act of measuring at least one property includes an act of measuring an impedance characteristic of at least one of the coils in the array.

9. The method of claim 8, wherein the act of measuring an impedance characteristic includes an act of obtaining a measured impedance matrix of the array of coils.

10. The method of claim 9, wherein the act of obtaining the measured impedance matrix includes an act of obtaining a plurality of scattering parameters (S-parameters) of the array of coils.

11. The method of claim 7, wherein the act of measuring at least one property includes an act of providing at least one electrical stimulus to at least one of the coils in the array.

12. The method of claim 11, wherein the act of providing at least one electrical stimulus includes an act of providing at least one of a current and a voltage to the at least one coil.

13. The method of claim 11, wherein the act of providing the electrical stimulus includes an act of providing an electrical stimulus having a range of frequencies and the act of measuring the at least one property includes an act of measuring at least one S-parameter of the array of coils.

14. The method of claim 13, wherein the act of measuring the at least one S-parameter includes an act of measuring a voltage in the at least one other of the coils in the array.

15. The method of claim 13, wherein the act of measuring at least one S-parameter includes an act of providing the electrical stimulus in one of the coils in the array and measuring the at least one property in each of the coils in the array.

16. The method of claim 15, wherein the act of measuring at least one S-parameter includes an act of measuring a plurality of S-parameters in part by producing a current in each of the coils in the array and measuring a voltage in each of the coils in the array, respectively, in response to the current.

17. The method of claim 16, wherein the act of measuring at least one property includes an act of obtaining a measured impedance matrix formed from the plurality of S-parameters.

18. The method of claim 8, wherein the act of determining at least one electromagnetic property includes an act of computing a trial impedance matrix from trial values of at least one of conductivity, permittivity and permeability for the at least one region of the body.

19. The method of claim 18, wherein the act of computing a trial impedance matrix includes computing values of the trial impedance matrix by solving Maxwell's equations with the trial values.

20. The method of claim 19, wherein the act of computing a trial impedance matrix includes computing the trial impedance matrix according to the expression:
$${Z}_{ij} = {\int }_{V} \{ σ ( x ) {\overline{E}}_{i}^{*} ( x ) \cdot {\overline{E}}_{j} ( x ) - ⅈω [ ɛ ( x ) {\overline{E}}_{i}^{*} ( x ) \cdot {\overline{E}}_{j} ( x ) - {μ ( x )}^{- 1} {\overline{B}}_{i}^{*} ( x ) \cdot {\overline{B}}_{j} ( x ) ] \} + {\int }_{S} {\overline{E}}_{i} ( x ) \times {\overline{B}}_{j}^{*} ( x ) \cdot ⅆ \overline{S} .$$

21. The method of claim 19, wherein the act of computing the trial impedance matrix includes employing a finite difference time domain (FDTD) simulation of a model of the array and the body to compute a plurality of currents flowing in a plurality of coils in the array in response to a plurality of voltages and computing impedance characteristics from the plurality of currents and the plurality of voltages.

22. The method of claim 18, wherein the act of determining at least one electromagnetic property includes an act of comparing the trial impedance matrix with the measured impedance matrix.

23. The method of claim 21, wherein the act of determining at least one electromagnetic property includes an act of reducing a distance between the trial impedance matrix and the measured impedance matrix.

24. The method of claim 23, wherein the act of reducing the distance includes iteratively updating the trial impedance matrix by updating trial values that decrease the distance from the measured impedance matrix to provide a final trial impedance matrix.

25. The method of claim 24, wherein the act of reducing the distance includes an act of determining a least squares distance.

26. The method of claim 24, wherein the act of determining at least one electromagnetic property includes an act of forming an image of the body, the image having a plurality of voxels, each voxel of the plurality of voxels having an intensity based on corresponding trial values used to compute the final trial impedance matrix.

27. The method of claim 8, wherein the act of determining at least one electromagnetic property includes providing a model of the array of coils and the body.

28. The method of claim 27, wherein the act of providing a model includes an act of logically partitioning a volume of space including at least a portion of the body into a plurality of regions.

29. The method of claim 28, wherein the act of determining at least one magnetic property includes an act of assigning at least one of a conductivity value, a permittivity value, and a permeability value to each of the plurality of regions.

30. The method of claim 29, wherein the act of determining at least one electromagnetic property includes an act of computing a trial impedance matrix from the assigned conductivity, permittivity and permeability values according to the model.

31. The method of claim 30, wherein the act of determining at least one electromagnetic property includes an act of reducing a distance between the trial impedance matrix and the measured impedance matrix by iteratively adjusting trial values of the assigned conductivity and permittivity values

32. The method of claim 31, wherein the act of computing the trial impedance matrix includes an act of performing a finite difference time domain simulation of the model.

33. A method of determining one or more properties of a body, the method comprising acts of:
positioning the body proximate a plurality of coils;
measuring at least one property of at least one of the plurality of coils; and
determining at least one electromagnetic property of at least one region of the body from the at least one property based on at least two of a resistive coupling, a capacitive coupling, and an inductive coupling between at least two of the plurality of coils.

34. The method of claim 33, wherein the act of determining at least one electromagnetic property includes an act of determining at least one of a conductivity, a permittivity, and a permeability of the at least one region of the body.

35. The method of claim 33, wherein the act of determining the at least one electromagnetic property includes an act of determining at least one of a magnitude, a direction, and a phase of an electric field at the at least one region of the body.

36. The method of claim 33, wherein the act of determining the at least one electromagnetic property includes an act of determining at least one of a magnitude, a direction, and a phase of a magnetic field at the at least one region of the body.

37. The method of claim 33, further comprising an act of forming an image having a plurality of voxels, each voxel of the plurality of voxels having an intensity related to a respective one of the at least one electromagnetic property.

38. The method of claim 33, wherein the act of measuring at least one property includes an act of measuring an impedance of at least one of the plurality of coils.

39. The method of claim 3 8, wherein the act of measuring an impedance includes an act of obtaining a measured impedance matrix of the plurality of coils.

40. The method of claim 39, wherein the act of measuring an impedance matrix includes measuring at least one scattering parameter (S-parameter) of at least one of the plurality of coils.

41. The method of claim 40, wherein the act of measuring at least one S-parameter includes an act of providing a current in at least one of the plurality of coils and measuring the at least one property in at least one other of the plurality of coils.

42. The method of claim 41, wherein the act of measuring the at least one property includes an act of measuring a voltage in the at least one other of the plurality of coils.

43. The method of claim 42, wherein the act of measuring the at least one property includes an act of measuring an S1 1 parameter of the at least one other of the plurality of coils.

44. The method of claim 41, wherein the act of measuring at least one S-parameter includes an act of providing the current in one of the plurality coils and measuring the at least one property in each other of the plurality of coils.

45. The method of claim 44, wherein the act of measuring at least one S-parameter includes an act of measuring a plurality of S-parameters in part by producing current in each of the plurality of coils and measuring a voltage in each other of the plurality of coils, respectively, in response to the current.

46. The method of claim 39, wherein the act of determining at least one electromagnetic property includes computing a trial impedance matrix from trial values of at least one of conductivity and permittivity for the at least one region of the body.

47. The method of claim 46, wherein the act of computing the trial impedance matrix includes computing values of the trial impedance matrix by solving Maxwell's equations in part with the trial values.

48. The method of claim 46, wherein the act of computing the trial impedance matrix includes computing the trial impedance matrix according to the expression:
$${Z}_{ij} = {\int }_{V} \{ σ ( x ) {\overline{E}}_{i}^{*} ( x ) \cdot {\overline{E}}_{j} ( x ) - ⅈω [ ɛ ( x ) {\overline{E}}_{i}^{*} ( x ) \cdot {\overline{E}}_{j} ( x ) - {μ ( x )}^{- 1} {\overline{B}}_{i}^{*} ( x ) \cdot {\overline{B}}_{j} ( x ) ] \} + {\int }_{S} {\overline{E}}_{i} ( x ) \times {\overline{B}}_{j}^{*} ( x ) \cdot ⅆ \overline{S} .$$

49. The method of claim 19, wherein the act of computing the trial impedance matrix includes employing a finite difference time domain (FDTD) simulation of a model of the array and the body to compute a plurality of currents flowing in a plurality of coils in the array in response to a plurality of voltages and computing impedance characteristics from the plurality of currents and the plurality of voltages.

50. The method of claim 46, wherein the act of determining at least one electromagnetic property includes an act of comparing the trial impedance matrix with the measured impedance matrix.

51. The method of claim 49, wherein the act of determining at least one electromagnetic property includes an act of reducing a distance between the trial impedance matrix and the measured impedance matrix.

52. The method of claim 51, wherein the act of reducing the distance includes iteratively updating the trial impedance matrix with update trial values that decrease the distance from the measured impedance matrix to provide a final trial impedance matrix.

53. The method of claim 52, wherein the act of reducing the distance includes an act of iteratively determining a least squares distance.

54. The method of claim 52, wherein the act of determining at least one electromagnetic property includes an act of forming an image of the body, the image having a plurality of voxels, each voxel of the plurality of voxels having an intensity based on corresponding updated trial values used to compute the final trial impedance matrix.

55. The method of claim 39, wherein the act of determining at least one electromagnetic property includes providing a model of the array of coils and the body.

56. The method of claim 55, wherein the act of providing a model includes an act of logically partitioning a volume of space including at least a portion of the body into a plurality of regions.

57. The method of claim 56, wherein the act of determining at least one electromagnetic property includes an act of assigning a conductivity value and a permittivity value to each of the plurality of regions.

58. The method of claim 57, wherein the act of determining at least one electromagnetic property includes an act of computing a trial impedance matrix from the assigned conductivity and permittivity values according to the model.

59. The method of claim 58, wherein the act of determining at least one electromagnetic property includes an act of reducing a distance between the trial impedance matrix and the measured impedance matrix by iteratively adjusting the values of the assigned conductivity and permittivity values.

60. The method of claim 59, wherein the act of computing the trial impedance matrix includes an act of performing a finite difference time domain simulation of the model.

61. An apparatus for determining one or more properties of a body, the apparatus comprising:
a plurality of coils having one or more resonant properties;
a first component coupled to the plurality of coils and adapted to provide at least one measurement of the plurality of coils indicative of a change in at least one resonant property of at least one of the plurality of coils; and
a second component coupled to the first component to receive the at least one measurement, the second component adapted to determine at least one electromagnetic property of at least one region of the body based on the change in the at least one resonant property.

62. The apparatus of claim 61, wherein the at least one resonant property includes at least one resonant frequency of at least one of the coils in the array.

63. The apparatus of claim 61, wherein the at least one electromagnetic property includes at least one of a conductivity, a permittivity, and a permeability of the at least one region of the body.

64. The apparatus of claim 61, wherein the at least one electromagnetic property includes at least one of a magnitude, a direction, and a phase of an electric field at the at least one region of the body.

65. The apparatus of claim 61, wherein the at least one electromagnetic property includes at least one of a magnitude, a direction, and a phase of a magnetic field at the at least one region of the body.

66. The apparatus of claim 61, wherein the second component is adapted to form an image having a plurality of voxels, each voxel of the plurality of voxels having an intensity related to a respective one of the at least one electromagnetic property.

67. The apparatus of claim 61, wherein the first component is adapted to measure an impedance of at least one of the plurality of coils.

68. The apparatus of claim 62, wherein the first component is adapted to obtain a measured impedance matrix of the plurality of coils.

69. The apparatus of claim 68, wherein the first component is adapted to measure at least one scattering parameter (S-parameter) of at least one of the plurality of coils.

70. The apparatus of claim 61, wherein the first component includes at least one of a matching circuit and a network analyzer.

71. The apparatus of claim 69, further comprising a third component adapted to provide a current in at least one of the plurality of coils and the first component is adapted to measure the at least one property in at least one other of the plurality of coils in response to the current.

72. The apparatus of claim 71, wherein the third component includes an radio frequency (RF) power source.

73. The apparatus of claim 71, wherein the first component is adapted to measure a voltage in the at least one other of the plurality of coils in response to the current.

74. The apparatus of claim 73, wherein the first component is adapted to measure an S11 parameter of the at least one other of the plurality of coils at a plurality of frequencies.

75. The apparatus of claim 68, wherein the first component is adapted to measure the at least one property in each of the other coils in the array in response to the current.

76. The apparatus of claim 68, wherein the second component is adapted to compute a trial impedance matrix from trial values of at least one of conductivity and permittivity for the at least one region of the body.

77. The apparatus of claim 76, wherein the second component is adapted to compute values of the impedance matrix by solving Maxwell's equations in part with the trial values.

78. The apparatus of claim 76, wherein the second component computes the trial impedance matrix according to the expression:
$${Z}_{ij} = {\int }_{V} \{ σ ( x ) {\overline{E}}_{i}^{*} ( x ) \cdot {\overline{E}}_{j} ( x ) - ⅈω [ ɛ ( x ) {\overline{E}}_{i}^{*} ( x ) \cdot {\overline{E}}_{j} ( x ) - {μ ( x )}^{- 1} {\overline{B}}_{i}^{*} ( x ) \cdot {\overline{B}}_{j} ( x ) ] \} + {\int }_{S} {\overline{E}}_{i} ( x ) \times {\overline{B}}_{j}^{*} ( x ) \cdot ⅆ \overline{S} .$$

79. The apparatus of claim 76, wherein the second component is adapted to compare the trial impedance matrix with the measured impedance matrix.

80. The apparatus of claim 79, wherein the second component determines at least one electromagnetic property in part by reducing a distance between the trial impedance matrix and the measured impedance matrix.

81. The apparatus of claim 80, wherein the second component reduces the distance in part by iteratively updating the trial impedance matrix with updated trial values that decrease the distance from the measured impedance matrix to provide a final trial impedance matrix.

82. The apparatus of claim 81, wherein the second component determines a least squares distance between the trial impedance matrix and the measured impedance matrix.

83. The apparatus of claim 81, wherein the second component is adapted to form an image having a plurality of voxels, each voxel of the plurality of voxels having an intensity corresponding to one of the updated trial values used to compute the final trial impedance matrix.

84. The apparatus of claim 68, wherein the second component provides a model of the plurality of coils and the body.

85. The apparatus of claim 84, wherein the model includes a logically partitioned volume of space having a plurality of regions, the plurality of regions including at least a portion of the body.

86. The apparatus of claim 85, wherein each of the plurality of regions is assigned a conductivity value and a permittivity value.

87. The apparatus of claim 86, wherein the second component computes a trial impedance matrix from the assigned conductivity and permittivity values according to the model.

88. The apparatus of claim 87, wherein the second component reduces a distance between the trial impedance matrix and the measured impedance matrix by iteratively adjusting trial values of the assigned conductivity and permittivity values.

89. The apparatus of claim 87, wherein the trial impedance matrix is computed by an finite difference time domain simulation of the model.

90. The apparatus of claim 61, wherein the second component includes:
at least one computer readable medium encoded with instructions; and
at least one processor coupled to the at least one computer readable medium, the at least one processor configured to execute the instructions.

91. An apparatus for determining one or more properties of a body, the apparatus comprising:
a plurality of coils;
a first component coupled to the plurality of coils, the first component adapted to provide at least one measurement of at least one property of the plurality of coils; and
a second component coupled to the first component to receive the at least one measurement, the second component adapted to determine at least one electromagnetic property of at least one region of the body from the at least one measurement based on at least two of a resistive coupling, a capacitive coupling, and an inductive coupling between two or more of the plurality of coils.

92. The apparatus of claim 91, wherein the at least one electromagnetic property includes at least one of a conductivity, a permittivity and a permeability of the at least one region of the body.

93. The apparatus of claim 91, wherein the at least one electromagnetic property includes at least one of a property of at least one of a magnetic and electric at the at least one region of the body.

94. The apparatus of claim 91, wherein the first component is adapted to measure an impedance of at least one of the plurality of coils.

95. The apparatus of claim 94, wherein the first component is adapted to obtain a measured impedance matrix of the plurality of coils.

96. The apparatus of claim 95, wherein the first component is adapted to measure at least one scattering parameter (S-parameter) of at least one of the plurality of coils.

97. The apparatus of claim 96, wherein the first component includes at least one of a matching circuit and a network analyzer.

98. The apparatus of claim 95, further comprising a third component adapted to provide a current in at least one of the plurality of coils and the first component is adapted to measure the at least one property in at least one other of the plurality of coils in response to the current.

99. The apparatus of claim 98, wherein the third component includes an radio frequency (RF) power source.

100. The apparatus of claim 98, wherein the first component is adapted to measure a voltage in the at least one other of the plurality of coils in response to the current.

101. The apparatus of claim 95, wherein the first component is adapted to provide at least one measurement of the at least one property in each of the other coils in the array in response to the current.

102. The apparatus of claim 95, wherein the second component is adapted to compute a trial impedance matrix from trial values of at least one of conductivity, permittivity, and permeability for the at least one region of the body.

103. The apparatus of claim 102, wherein the second component is adapted to compute values of the impedance matrix by solving Maxwell's equations in part with the trial values.

104. The apparatus of claim 102, wherein the second component computes the trial impedance matrix according to the expression:
$${Z}_{ij} = {\int }_{V} \{ σ ( x ) {\overline{E}}_{i}^{*} ( x ) \cdot {\overline{E}}_{j} ( x ) - ⅈω [ ɛ ( x ) {\overline{E}}_{i}^{*} ( x ) \cdot {\overline{E}}_{j} ( x ) - {μ ( x )}^{- 1} {\overline{B}}_{i}^{*} ( x ) \cdot {\overline{B}}_{j} ( x ) ] \} + {\int }_{S} {\overline{E}}_{i} ( x ) \times {\overline{B}}_{j}^{*} ( x ) \cdot ⅆ \overline{S} .$$

105. The apparatus of claim 102, wherein the second component is adapted to compare the trial impedance matrix with the measured impedance matrix.

106. The apparatus of claim 105, wherein the second component determines at least one electromagnetic property in part by reducing a distance between the trial impedance matrix and the measured impedance matrix.

107. The apparatus of claim 106, wherein the second component reduces the distance in part by iteratively updating the trial impedance matrix in a direction that decreases the distance from the measured impedance matrix.

108. The apparatus of claim 107, wherein the second component determines a least squares distance between the trial impedance matrix and the measured impedance matrix.

109. The apparatus of claim 107, wherein the second component is adapted to form an image having a plurality of voxels, each voxel of the plurality of voxels having an intensity corresponding to one of the updated trial values used to compute the final trial impedance matrix.

110. The apparatus of claim 95, wherein the second component provides a model of the plurality of coils and the body.

111. The apparatus of claim 110, wherein the model includes a logically partitioned volume of space having a plurality of regions, the plurality of regions including at least a portion of the body.

112. The apparatus of claim 111, wherein each of the plurality of regions is assigned a conductivity value and a permittivity value.

113. The apparatus of claim 112, wherein the second component computes a trial impedance matrix from the assigned conductivity and permittivity values according to the model.

114. The apparatus of claim 112, wherein the second component reduces a distance between the trial impedance matrix and the measured impedance matrix by iteratively adjusting trial values of the assigned conductivity and permittivity values.

115. The apparatus of claim 112, wherein the trial impedance matrix is computed by an finite difference time domain simulation of the model.

116. The apparatus of claim 91, wherein the second component includes:
at least one computer readable medium encoded with instructions; and
at least one processor coupled to the at least one computer readable medium, the at least one processor configured to execute the instructions.

117. A computer readable medium encoded with instructions capable of being executed on at least one processor, the instructions, when executed by the at least one processor, performing a method of determining one or more properties of a body positioned proximate a coil array, the method comprising acts of:
defining an electromagnetic model of the coil array;
receiving an input including a measured impedance matrix of the coil array;
logically partitioning a volume associated with the model of the coil array and the body into a plurality of regions;
assigning trial values respectively to each of the plurality of regions, the trial values including at least one of conductivity, permittivity and permeability;
generating a trial impedance matrix from the assigned trial values according to the electromagnetic model of the coil array; and
reducing a distance between the trial impedance matrix and the measured impedance matrix.

118. The computer readable medium of claim 117, wherein the act of generating the trial impedance matrix includes an act of generating the trial impedance matrix by implementing a finite difference time domain simulation of the model.

119. The computer readable medium of claim 117, wherein the act of reducing the distance includes determining a least squares distance between the trial impedance matrix and the measured impedance matrix by iteratively updating the conductivity and permittivity values such that the trial impedance matrix is closer to the measured impedance matrix on each iteration.
